(19) United States
(12) Patent Application Publication
(10) Pub. No.: US 2023/0135552 A1
(43) Pub. Date: May 4, 2023

(54) Compounds Useful In Inhibiting Ketohexokinase And Methods Of Making And Using The Same

(71) Applicant: Inorbit Therapeutics AB, Billdal (SE)

(72) Inventors: Lambertus Benthem, Billdal (SE); Robert Judkins, Molndal (SE)

(73) Assignee: Inorbit Therapeutics AB, Billdal (SE)

(21) Appl. No.: 17/798,702

(22) PCT Filed: Feb. 11, 2021
(86) PCT No.: PCT/GB2021/050326

§ 371 Date: Aug. 10, 2022

(30) Foreign Application Priority Data
Feb. 11, 2020 (GB) 2001856.0

(51) Int. Cl. C07D 417/14; A61P 3/10

(52) U.S. Cl. CPC C07D 417/14; A61P 3/10

(57) ABSTRACT

The present invention relates to compounds that can act as inhibitors of ketohexokinase (KHK) and that can be useful in the treatment of diseases and/or disorders associated with KHK. In some embodiments, the present invention relates to compounds and compositions that inhibit KHK and methods for their preparation and use.

### FIELD

The present invention relates to compounds that can act as inhibitors of ketohexokinase (KHK) and that can be useful in the treatment of diseases and/or disorders associated with KHK. In some embodiments. the present invention relates to compounds and compositions that inhibit KHK and methods for their preparation and use.

### BACKGROUND

Dramatic increases in fructose consumption and its metabolism could be a dietary factor contributing to nonalcoholic fatty liver disease (NAFLD), dyslipidemia, obesity, and insulin resistance in humans. Fructose has been shown to cause fat accumulation in the liver via increased lipogenesis and impaired fat oxidation. In addition, it is preferentially metabolised over other carbohydrates to produce various reactive and signaling metabolites that contribute to metabolic disease progression.

Increases in liver fat have been shown to occur on administration of fructose-containing beverages in a clinical setting, suggesting that fructose has a role in NAFLD. In addition, studies have shown a correlation in a dose dependent manner of such sugary drinks with the severity of liver cirrhosis.

Fructose is principally metabolised by the fructokinase enzyme ketohexokinase (KHK). KHK phosphorylates the sugar to fructose-1-phosphate (F1P), which in turn is converted to D-glyceraldehyde and dihydroxyacetone phosphate by aldolase B. From here on the metabolism is similar to that of glucose with the end result being the formation of fatty acids and triglycerides.

However, the initial phosphorylation of fructose by KHK occurs without any feedback control and leads to a significant drop in cellular ATP levels, significantly differentiating this pathway from glucose metabolism. The depletion of ATP on ingestion of fructose has been shown in humans and also results in an increase in uric acid levels due to the activation of adenosine monophosphate (AMP) deaminase which converts AMP to inosine monophosphate IMP (a precursor for the biosynthesis of uric acid).

KHK exits as two isoforms, KHK-A and KHK-C. The former being expressed ubiquitously whilst the latter is found in the liver, kidney and intestine.

KHK-C has a higher efficiency than KHK-A in the phosphorylation of fructose, driving the fall in intracellular phosphate levels and the fructose induced fatty liver observed in animal models. It has been shown that mice lacking both fructokinase C and A are protected from fructose-induced fatty liver and that fructose, but not glucose, drives lipogenic enzymes and insulin resistance, through fructokinase, and that fructokinase levels are elevated in fructose fed mice as well as obese humans with NASH.

Inhibition of KHK therefore has the potential to be a treatment for a range of diseases including diabetes (T1D and/or T2D), idiopathic T1D (Type 1b), latent autoimmune diabetes in adults (LADA), early-onset T2D (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, obesity, eating disorders, excessive sugar craving, dyslipidemia (including hyperlipidemia, hypertriglyceridemia, increased total cholesterol, high LDL cholesterol, and low HDL cholesterol), hyperinsulinemia, NAFLD (including related diseases such as steatosis, NASH, fibrosis, cirrhosis, and hepatocellular carcinoma), HFI, coronary artery disease, peripheral vascular disease, hypertension, endothelial dysfunction, impaired vascular compliance, congestive heart failure, myocardial infarction (e.g. necrosis and apoptosis), stroke, hemorrhagic stroke, ischemic stroke, pulmonary hypertension, restenosis after angioplasty, intermittent claudication, post-prandial lipemia, metabolic acidosis, ketosis, arthritis, osteoporosis, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, foot ulcerations, ulcerative colitis, hyper apo B lipoproteinemia, Alzheimer's Disease, schizophrenia, impaired cognition, inflammatory bowel disease, ulcerative colitis, Crohn's disease, and irritable bowel syndrome.

Although several KHK inhibitors are known (Inhibitors of Ketohexokinase: Discovery of Pyrimidinopyrimidines with Specific Substitution that Complements the ATP-Binding Site, ACS Med Chem Lett. 2011 Jul. 14; 2(7): 538-543; Discovery of Fragment-Derived Small Molecules for in Vivo Inhibition of Ketohexokinase (KHK), J Med Chem. 2017 Sep. 28; 60(18):7835-7849) there is still a need for the development of novel and potent compounds for the treatment and prevention of disease,

The listing or discussion of an apparently prior-published document in this specification should not necessarily be taken as an acknowledgement that the document is part of the state of the art or is common general knowledge.

### SUMMARY

The present inventors have surprisingly and unexpectedly found that the presence of a sulfinic and/or sulfonic group on compounds that are KHK inhibitors can alter the metabolism pathway resulting in a decrease in harmful metabolites formed, particularly via acyl-glucuronidation without significant loss of efficacy as KHK inhibitors,

The present invention relates to the compounds defined by Formula A (as well as Formulae A1, B, B1, B2, B3, C and C1) as detailed below, which are referred to hereinafter as “the compounds of the invention”.

A first aspect of the present invention is directed to a compound having a structure represented by Formula A:

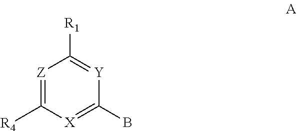

wherein

Z is CH, N or C—CN;

Y is N or CH;

X is N or CR₃;

B is

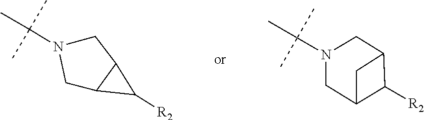

R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein said —C₁₋₃ alkyl is substituted with 0 to 3 halogen atoms, and provided that there is no more than one —OH substituent; or

R₁ is —N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;

R₂ is -(L)ₘ—S(O)OH, -L-(CH₂)ₙS(O)OH, -(L)ₘ-S(O)₂OH, -L-(CH₂)ₙS(O)₂OH;

m is 0 or 1:

n is 0 or 1;

L is CH₂, CHF, or CF₂;

R₃ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;

R₄ is —C₁₋₃ alkyl or —C₃₋₄ cycloalkyl, wherein the C₁₋₃ alkyl or —C₃₋₄ cycloalkyl group is is optionally substituted with 0 to 5 halogen atoms;

or R₃ and R₄ come together with the carbon atoms to which they are attached to form a C₄₋₇ cycloalkyl ring wherein each carbon atom in the ring is substituted by 0 to 2 R₅ groups;

R₅ is F, —C₁₋₃ alkyl, —OC₁₋₃ alkyl, —C₃₋₄ cycloalkyl, wherein each O₁₋₃ alkyl or —C₃₋₄ cycloalkyl is optionally substituted with 1 to 3 halogen atoms;

or an enantiomer, stereoisomer, tautomer, solvate, hydrate, prodrug, amino acid conjugate, metabolite, or pharmaceutically acceptable salt thereof.

A second aspect of the present invention is directed to a compound having a structure represented by Formula B:

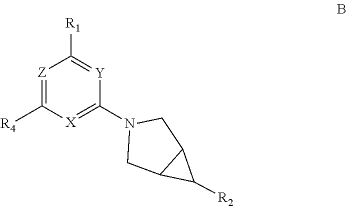

wherein

Z is CH, N or C—CN;

Y is N or CH;

X is N or CR₃;

R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or

—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;

R₂ is -(L)ₘ-S(O)OH, -L-(CH₂)ₙS(O)OH, -(L)ₘ-S(O)₂OH, -L-(CH₂)ₙS(O)₂OH;

m is 0 or 1;

n is 0 or 1;

L is CH₂, CHF, or CF₂;

R₃ is H, halogen, —CN, —C₁₋₃ alkyl, —OC₁₋₃ alkyl, —C₃₋₄ cycloalkyl, wherein each —C₁₋₃ alkyl or —C₃₋₄ cycloalkyl is optionally substituted with 1 to 3 halogen atoms; and

R₄ is cyclopropyl, cyclobutyl, or —C₁₋₃ alkyl, wherein the C₁₋₃ alkyl is optionally substituted with 0 to 5 halogen atoms;

or an enantiomer, stereoisomer, tautomer, solvate, hydrate, prodrug, amino acid conjugate, metabolite, or pharmaceutically acceptable salt thereof.

A third aspect of the present invention is directed to a compound having a structure represented by Formula C:

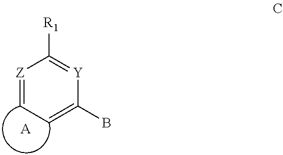

wherein

Z is CH, N or C—CN;

Y is N or CH;

Ring A is a C₄₋₇ cycloalkyl ring wherein each carbon atom in the ring is substituted by 0 to 2 R₅ groups;

B is

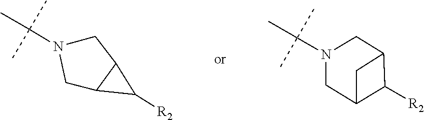

R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein said —C₁₋₃ alkyl is substituted with 0 to 3 halogen atoms, and provided that there is no more than one —OH substituent; or

R₁ is —N(C₁₋₃alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;

R₂ is -(L)ₘ-S(O)OH, -L-(CH₂)ₙS(O)OH, -(L)ₘ-S(O)₂OH, -L-(CH₂)ₙS(O)₂OH;

m is 0 or 1;

n is 0 or 1;

L is CH₂, CHF, or CF₂;

R₅ is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;

or an enantiomer, stereoisomer, tautomer, solvate, hydrate, prodrug, amino acid conjugate, metabolite, or pharmaceutically acceptable salt thereof.

A fourth aspect of the present invention is directed to a pharmaceutical (or veterinary) is composition comprising a compound of the invention (i.e. a compound of Formula A, A1, B, B1, B2, B3, C or C1 or an enantiomer, stereoisomer, tautomer, solvate, hydrate, prodrug, amino acid conjugate, metabolite, or pharmaceutically acceptable salt thereof) and a pharmaceutically acceptable excipient, diluent and/or carrier, which are referred to hereinafter as “compositions of the invention”.

Another aspect of the present invention is directed to a method of inhibiting ketohexokinase (KHK), the method comprising administering to a subject in need thereof an effective amount of a compound of the invention (i.e. a compound of Formula A, A1, B, B1, B2, B3, C or C1 or an enantiomer, stereoisomer, tautomer, solvate, hydrate, prodrug, amino acid conjugate, metabolite, or pharmaceutically acceptable salt thereof) or a composition of the invention.

The present invention is also directed to a compound of the invention or a composition of the invention for use in inhibiting KHK. The present invention is also directed to the use of a compound of the invention or a composition of the invention in the manufacture of a medicament for inhibiting KHK.

The present invention is also directed to a method of treating and/or preventing a disease or disorder in which ketohexokinase (KHK) plays a role, the method comprising administering to a subject in need thereof an effective amount of a compound of the invention or a composition of the invention. Still further, the invention is directed to the use of a compound of the invention or a composition of the invention, in the manufacture of a medicament for treating and/or preventing a disease or disorder in which ketohexokinase (KHK) plays a role.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner, These and other objects and/or aspects of the present invention are explained in detail in the specification set forth below. Further features, advantages and details of the present invention will be appreciated by those of ordinary skill in the art from a reading of the figure and the detailed description of the preferred embodiments that follow, such description being merely illustrative of the is present invention.

### DETAILED DESCRIPTION

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention, All publications, patent applications, patents and other references mentioned herein are incorporated by reference in their entirety. In case of a conflict in terminology, the present specification is controlling.

The terminology used in the description of the invention herein is for the purpose of describing particular embodiments only and is not intended to be limiting of the invention, As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, is “about X” where X is the measurable value, is meant to include X as well as variations of ±10%, ±5%, ±1%, ±0.5%, or even ±0.1% of X. A range provided herein for a measureable value may include any other range and/or individual value therein.

“Ketohexokinase” or “KHK” as used herein is ketohexokinase from any source and/or that is present in a subject and/or expressed in any form. In some embodiments, ketohexokinase is from and/or is present and/or expressed in an animal such as, e.g., a mammal. In some embodiments, ketohexokinase is from and/or is present and/or expressed in a primate, cow, sheep, goat, horse, dog, cat, rabbit, rat, mouse, fish, bird, and/or the like. In some embodiments, ketohexokinase is from and/or is present and/or expressed in a human.

The term “inhibit”, in reference to KHK, refers to the ability of a compound (e.g., a compound of the present invention) to inhibit one or more function(s), action(s), and/or characteristic(s) of KHK, either directly or indirectly and may occur in-vitro or in-vivo.

The term “inhibitor” refers to a compound (e.g., a compound of the present invention) that combines with and/or binds to a specific enzyme (e.g. KHK) and decreases a function, action, and/or characteristic associated with the enzyme. In some embodiments, a compound of the present invention is a KHK inhibitor.

“Substantially the same” as used herein in reference to a measurable value and/or response means being within about ±10% of the compared to value and/or response.

The term “C₁₋₃ alkyl” means a saturated or unsaturated alkyl chain having 1 to 3 carbon atoms which may be a straight chain or branched chain. Examples thereof include, but are not limited to, methyl, ethyl, propyl and isopropyl.

The term “C₃₋₄ cycloalkyl” means a saturated monocyclic ring system comprising 3 to 4 carbon atoms. Examples thereof are cyclopropyl and cyclobutyl.

The term “C₃₋₇ cycloalkyl” means a saturated mono-, bi-, spiro- or multicyclic ring system comprising 3 to 7 carbon atoms. Examples thereof include, but are not limited to, cyclopropyl, cyclobutyl, cyclopentyl, cyclohexyl, cycloheptyl, bicyclo[3.2.0]heptane, spiro[2.3]hexane, spiro[2.4]heptane and spiro[3.3]heptane.

The term “4- to 7-membered heterocyclic” means a saturated mono-, bi-, spiro- or multicyclic ring system that contains 4- to 7 atoms selected from carbon, nitrogen, oxygen, and/or sulfur with 1 to 2 of those atoms being a heteroatom (i.e., nitrogen, oxygen, and/or sulfur). Examples thereof include, but are not limited to, pyrrolidine, thiolane, tetrahydrofuran, piperidine, tetrahydropyran, thiane, morpholine, piperazine, and dioxane. If not stated otherwise, a 4- to 7-membered mono-, bi-, spiro- or multicyclic ring system as described herein can be connected via a carbon or nitrogen atom.

“Halogen” refers to fluorine, chlorine, bromine and iodine. In some embodiments, the halogen is fluorine or chlorine.

The term “sulfinic acid” means the functional group S(O)OH, consisting of a sulfinyl group and a hydroxyl group.

The term “sulfinate” means the conjugate base of sulfinic acid, where the hydroxyl has been deprotonated to give S(O)O⁻.

The term “sulfonic acid” means the functional group S(O)₂OH, consisting of a sulfonyl group and a hydroxyl group.

The term “sulfonate” means the conjugate base of sulfonic acid, where the hydroxyl has been deprotonated to give S(O)₂O⁻.

The term “optionally substituted” is understood to mean that a given chemical moiety (e.g., an alkyl group) can (but is not required to) be bonded to other substituents (e.g., heteroatoms). For instance, an alkyl group that is optionally substituted can be a fully saturated alkyl chain (e.g., a pure hydrocarbon). Alternatively, the same optionally substituted alkyl group can have one or more substituent(s) different from hydrogen, For instance, it can, at any point along the chain, be bound to a halogen atom, a hydroxyl group, or any other substituent described herein. Thus, the term “optionally substituted” means that a given chemical moiety has the potential to contain other functional groups but does not necessarily have any further functional groups.

The term “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

The term “pharmaceutically acceptable salt” refers to a salt of a compound which is, within the scope of sound medical judgment, suitable for use in contact with the tissues of humans and lower animals without undue toxicity, irritation, allergic response and the like, and is commensurate with a reasonable benefit/risk ratio, Pharmaceutically acceptable salts are well known in the art.

For a detailed review of pharmaceutically acceptable salts see J. Pharmaceutical Sciences, 66: 1-19 (1977), by Berge et al. In some embodiments, the salts can be prepared in situ during the final isolation and/or purification for a compound of the invention, or separately by reaction of the free acid function with a suitable inorganic or organic base. Suitable salts include, but are not limited to, metals, such as sodium, potassium and calcium, or amines, such as triethylammonium, ethanolammonium and lysine.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “prodrug” refers to a compound which is, within the scope of sound medical judgment, suitable for use in contact with the tissues of humans and lower animals with undue toxicity, irritation, allergic response, and/or the like, commensurate with a reasonable benefit/risk ratio, and effective for their intended use, as well as the zwitterionic forms, where possible, of a compound of the present invention. “Prodrug”, as used herein means a compound that is convertible in vivo by metabolic means (e.g., by hydrolysis) to afford a compound of the present invention (e.g., a compound of Formula A, A1, B, B1, B2, B3, C or C1).

Various forms of prodrugs are known in the art, for example, as discussed in Bundgaard, (ed.), Design of Prodrugs, Elsevier (1985); Widder, et al. (ed.), Methods in Enzymology, Vol. 4, Academic Press (1985); Krogsgaard-Larsen, et al,, (ed). “Design and Application of Prodrugs, Textbook of Drug Design and Development”, Chapter 5, 113-191 (1991), Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The term “amino acid conjugate” refers to a conjugate of a compound of the present invention (e.g., a compound of Formula A, A1, B, B1, B2, B3, C or C1) with an amino acid. Preferably, such amino acid conjugates of the present invention will have the added advantage of enhanced integrity in bile and/or intestinal fluids. Suitable amino acids include, but are not limited to, glycine and taurine. Thus, the present invention encompasses the glycine and taurine conjugates of a compound of Formulas A, A1, B, B1, B2, B3, C and C1.

Unless indicated otherwise, nomenclature used to describe chemical groups or moieties as used herein follow the convention where, reading the name from left to right, the point of attachment to the rest of the molecule is at the right-hand side of the name, For example, methylamino, where the point of attachment is at the amine end. In other circumstances the point of attachment is indicated by “−”, for example, —NH(C₁₋₃ alkyl) is attached at the nitrogen.

Unless indicated otherwise, where a mono or bivalent group is described by its chemical formula, including one or two terminal bond moieties indicated by “−,” it will be understood that the attachment is read from left to right.

Unless otherwise stated, structures depicted herein are meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

The present invention is directed to a compound having a structure represented by Formula A:

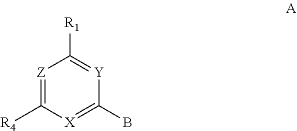

wherein

Z is CH, N or C—CN;

Y is N or CH;

X is N or CR₃;

B is

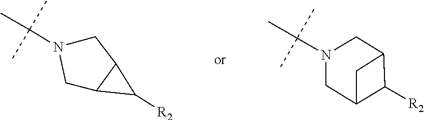

R₁ is C_{3- 7} cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein said —C₁₋₃ alkyl is substituted with 0 to 3 halogen atoms, and provided that there is no more than one —OH substituent; or

R₁ is —N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;

R₂ is -(L)ₘ-S(O)OH, -L-(CH₂)ₙS(O)OH, -(L)ₘ-S(O)₂OH, -L-(CH₂)ₙS(O)₂OH:

m is 0 or 1;

n is 0 or 1;

L is CH₂, CHF, or CF₂;

R₃ is H, halogen, —ON, —C₁₋₃ alkyl, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;

R₄ is —C₁₋₃ alkyl or —C₃₋₄ cycloalkyl, wherein the C₁₋₃ alkyl or −C₃₋₄ cycloalkyl group is optionally substituted with 0 to 5 halogen atoms;

or R₃ and R₄ come together with the carbon atoms to which they are attached to form a C₄₋₇ cycloalkyl ring wherein each carbon atom in the ring is substituted by 0 to 2 R₅ groups;

R₅ is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;

or an enantiomer, stereoisomer, tautomer, solvate, hydrate, prodrug, amino acid conjugate, metabolite, or pharmaceutically acceptable salt thereof.

In an embodiment of Formula A, the ring containing Y and Z is not fused to a second ring.

For example, the compounds may have a structure represented by Formula A1:

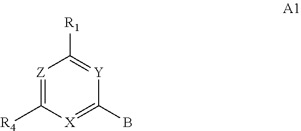

wherein

Z, Y, X, B and R₁ are as defined above in respect of Formula A;

R₃ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and

R₄ is —C₁₋₃ alkyl or —C₃₋₄ cycloalkyl, wherein the C₁₋₃ alkyl or —C₃₋₄ cycloalkyl group is optionally substituted with 0 to 5 halogen atoms;

(including enantiomers, stereoisomers, tautomers, solvates, hydrates, prodrugs, amino acid conjugates, metabolites, and pharmaceutically acceptable salts thereof).

In the second aspect, the invention provides a compounds having a structure represented by Formula B:

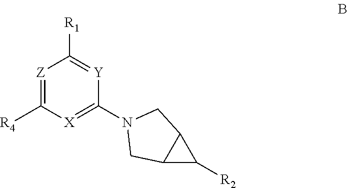

wherein

Z is CH, N or CCN;

Y is N or CH;

X is N or CR₃ₐ;

R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or

—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;

R₂ is -(L)ₘ-S(O)OH, -L-(CH₂)ₙS(O)OH, -(L)ₘ-S(O)₂OH, -L-(CH₂)ₙS(O)₂OH;

m is 0 or 1;

n is 0 or 1;

L is CH₂, CHF, or CF₂;

R₃ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and

R₄ is cyclopropyl, cyclobutyl, or —C₁₋₃ alkyl, wherein the C₁₋₃ alkyl is optionally substituted with 0 to 5 halogen atoms;

or an enantiomer, stereoisomer, tautomer, solvate, hydrate, prodrug, amino acid conjugate, metabolite, or pharmaceutically acceptable salt thereof.

In another embodiment, the compounds have a structure represented by Formula B1:

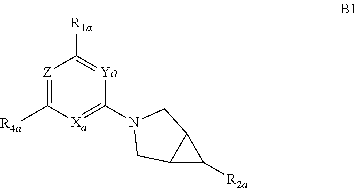

wherein

Y, is N or CH;

X, is N or CR₃ₐ;

R₁ₐ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or

R₁ₐ is —N(C₁₋₃alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;

R₂ₐ is -(L)ₘ-S(O)OH, -L-(CH₂)ₙS(O)OH, -(L)ₘ-S(O)₂OH, or -L-(CH₂)ₙS(O)₂OH;

m is 0 or 1;

n is 0 or 1;

L is CH₂, CHF, or CF₂:

R₃ₐ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and

R₄ₐ is cyclopropyl, cyclobutyl, or —C₁₋₃ alkyl, wherein the C₁₋₃ alkyl is optionally substituted with 0 to 5 halogen atoms;

or an enantiomer, stereoisomer, tautomer, solvate, hydrate, prodrug, amino acid conjugate, metabolite, or pharmaceutically acceptable salt thereof.

In a further embodiment, the compounds have a structure represented by Formula B2:

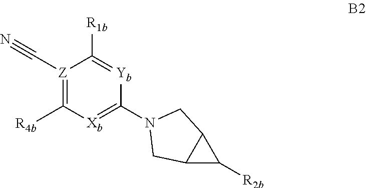

wherein

Y_{b} is N or CH;

X_{b} is N or CR_{3b};

R_{1b} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or

R_{1b} is —N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;

R_{2b} is -(L)ₘ-S(O)OH, -L-(CH₂)ₙS(O)OH, -(L)ₘ-S(O)₂OH or -L-(CH₂)ₙS(O)₂OH;

m is 0 or 1;

n is 0 or 1;

L is CH₂, CHF, or CF₂:

R_{3b} is H, halogen, —CN, —C₁₋₃ alkyl, —OC₁₋₃ alkyl, -C3.4 cycloalkyl, wherein each —C₁₋₃ alkyl or —C₃₋₄ cycloalkyl is optionally substituted with 1 to 3 halogen atoms; and

R_{4b} is cyclopropyl, cyclobutyl, or —C₁₋₃alkyl, wherein the C₁₋₃ alkyl is optionally substituted with 0 to 5 halogen atoms;

or an enantiomer, stereoisomer, tautomer, solvate, hydrate, prodrug, amino acid conjugate, metabolite, or pharmaceutically acceptable salt thereof.

In a yet further embodiment, the compounds have a structure represented by Formula B3:

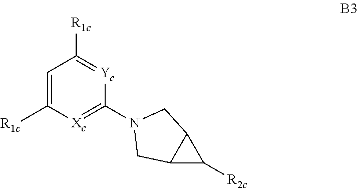

wherein

Y, is N or CH;

X, is N or CR_{3c};

R_{1c} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or

R_{1c} is —N(C₁₋₃ alkyl)₂, —NH(C₁₋₃ alkyl), or —NH(C₃₋₄cycloalkyl), wherein each C₁₋₃ alkyl or C₃₋₄ cycloalkyl is substituted with 0 to 1 OH;

R_{2c} is -(L)ₘ-S(O)OH, -L-(CH₂)ₙS(O)OH, -(L)ₘ-S(O)₂OH or -L-(CH₂)ₙS(O)₂OH;

m is 0 or 1;

n is 0 or 1;

L is CH₂, CHF, or CF₂;

R_{3c} is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and

R_{4c} is cyclopropyl, cyclobutyl, or —C₁₋₃alkyl, wherein the C₁₋₃ alkyl is optionally substituted with 0 to 5 halogen atoms;

or an enantiomer, stereoisomer, tautomer, solvate, hydrate, prodrug, amino acid conjugate, metabolite, or pharmaceutically acceptable salt thereof.

In one embodiment, the invention relates to a compound of Formula B1 (or an enantiomer, stereoisomer, tautomer, solvate, hydrate, prodrug, amino acid conjugate, metabolite, or pharmaceutically acceptable salt thereof) in which:

Yₐ is N;

Xₐ is CR₃ₐ;

R₁ₐ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent;

R₂ₐ is —CH₂—S(O)OH or —(CH₂)₂—S(O)OH;

R₃ₐ is H, halogen or —C₁₋₃ alkyl optionally substituted with 1 to 3 halogen atoms; and

R₄ₐ is —C₁₋₃ alkyl optionally substituted with 0 to 5 halogen atoms.

In another embodiment, the invention relates to a compound of Formula B1 (or an enantiomer, stereoisomer, tautomer, solvate, hydrate, prodrug, amino acid conjugate, metabolite, or pharmaceutically acceptable salt thereof) in which:

Yₐ is N;

Xₐ is CR₃ₐ;

R₁ₐ is a 4- to 7-membered heterocyclic moiety, wherein the heterocyclic moiety is bound via a nitrogen atom and optionally contains one further heteroatom in the ring selected from nitrogen, oxygen and sulfur, and wherein the heterocyclic moiety has 0 to 3 substituents independently selected from —C₁₋₃ alkyl and —OH, wherein said —C₁₋₃ alkyl is substituted with 0 to 3 halogen atoms, and provided that there is no more than one —OH substituent;

R₂ₐ is —CH₂—S(O)OH or —(CH₂)₂—S(O)OH;

R₃ₐ is H, halogen or —C₁₋₃ alkyl optionally substituted with 1 to 3 halogen atoms (e.g. R₃ₐ is H or methyl); and

R₄ₐ is —C₁₋₃ alkyl optionally substituted with 1 to 5 halogen atoms (e.g. a methyl group optionally substituted by 1 to 3 halogen atoms).

In another embodiment of Formula A, X is CR₃ and R₃ and R₄ come together with the carbon atoms to which they are attached to form a C₄₋₇ cycloalkyl ring wherein each carbon atom in the ring is substituted by 0 to 2 R₅ groups.

In the third aspect, the invention provides a compound having a structure represented by Formula C:

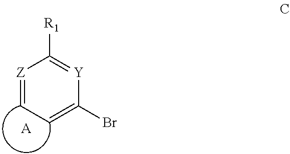

wherein

Z is CH, N or C—CN;

Y is N or CH;

Ring A is a C₄₋₇ cycloalkyl ring wherein each carbon atom in the ring is substituted by 0 to 2 R₅ groups;

B is

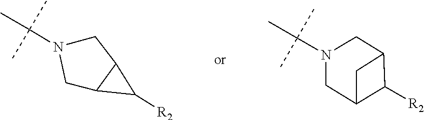

R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein said —C₁₋₃ alkyl is substituted with 0 to 3 halogen atoms, and provided that there is no more than one —OH substituent; or

R₁ is —N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;

R₂ is -(L)ₘ-S(O)OH, -L-(CH₂)ₙS(O)OH, -(L)ₘ-S(O)₂OH, -L-(CH₂)ₙS(O)₂OH:

m is 0 or 1;

n is 0 or 1;

L is CH₂, CHF, or CF₂;

R₅ is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;

or an enantiomer, stereoisomer, tautomer, solvate, hydrate, prodrug, amino acid conjugate, metabolite, or pharmaceutically acceptable salt thereof.

In a fourth aspect, the invention provides a compound having a structure represented by Formula C1:

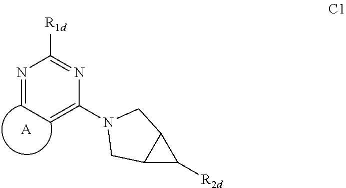

wherein

Ring A is a C₄₋₇ cycloalkyl ring wherein each carbon atom in the ring is substituted by 0 to 2 R_{5d} groups;

R_{1d} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH (e.g. selected from —C₁₋₃ alkyl and —OH), wherein said —C₁₋₃ alkyl is substituted with 0 to 3 halogen atoms, and provided that there is no more than one —OH substituent; or

R_{1d} is —N(C₁₋₃alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;

R_{2d} is -(L)ₘ-S(O)OH, -L-(CH₂)ₙS(O)OH, -(L)ₘ-S(O)₂OH, -L-(CH₂)ₙS(O)₂OH;

m is 0 or 1;

n is 0 or 1;

L is CH₂, CHF, or CF₂;

R_{5d} is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;

or an enantiomer, stereoisomer, tautomer, solvate, hydrate, prodrug, amino acid conjugate, metabolite, or pharmaceutically acceptable salt thereof.

In an embodiment of the compound of Formula C1:

Ring A is a C₅₋₆ cycloalkyl ring wherein each carbon atom in the ring is substituted by 0 to 2 R_{5d} groups;

R_{1d} is a 4- to 7-membered heterocyclic moiety, wherein the heterocyclic moiety is bound via a nitrogen atom and optionally contains one further heteroatom in the ring selected from nitrogen, oxygen and sulfur, and wherein the heterocyclic moiety has 0 to 3 substituents independently selected from —C₁₋₃ alkyl and —OH, wherein said —C₁₋₃ alkyl is substituted with 0 to 3 halogen atoms, and provided that there is no more than one —OH substituent;

R_{5d} is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms (e.g. R_{5d} is F or methyl);

In one embodiment of any of Formulae A, B and C, Z is CH. In another embodiment of any of Formulae A, B and C, Z is N. In a further embodiment of any of Formulae A, B and C, Z is C—CN.

Particular compounds of the invention that may be mentioned include;

sodium((1R,5S,6S)-3-(2-((S)-2-methylazetidin-1-yl)-6-(trifluoromethyl)pyrimidin-4-yl)-3-azabicyclo[3.1.0]hexan-6-yl)methanesulfinate;

((1R,5S,6S)-3-(2-((S)-2-methylazetidin-1-yl)-6-(trifluoromethyl)pyrimidin-4-yl)-3-azabicyclo[3.1.0]hexan-6-yl)methanesulfinic acid;

sodium((1R,5S,6S)-3-(2-((2S,3R)-3-hydroxy-2-methylazetidin-1-yl)-6-(trifluoromethyl)pyrimidin-4-yl)-3-azabicyclo[3.1.0]hexan-6-yl)methanesulfinate;

((1R,5S,6S)-3-(2-((2S,3R)-3-hydroxy-2-methylazetidin-1-yl)-6-(trifluoroethyl)pyrimidin-4-yl)-3-azabicyclo[3.1.0]hexan-6-yl)methanesulfinic acid;

Sodium((1R,5S,6S)-3-(2-((S)-2-methylpyrrolidin-1-yl)-6-(trifluoroethyl)pyrimidin-4-yl)-3-azabicyclo[3.1.0]hexan-6-yl)methanesulfinate;

((1R,5S,6S)-3-(2-((S)-2-methylpyrrolidin-1-yl)-6-(trifluoromethyl)pyrimidin-4-yl)-3-azabicyclo[3.1.0]hexan-6-yl)methanesulfinic acid;

Sodium((1R,5S,6S)-3-(2-((S)-2-methylpiperidin-1-yl)-6-(trifluoromethyl)pyrimidin-4-yl)-3-azabicyclo[3.1.0]hexan-6-yl)methanesulfinate;

((1R,5S,6S)-3-(2-((S)-2-methylpiperidin-1-yl)-6-(trifluoromethyl)pyrimidin-4-yl)-3-azabicyclo[3.1.0]hexan-6-yl)methanesulfinic acid;

Sodium((1R 5S ,6S)-3-(5-methyl-2-((S)-2-methylazetidin-1-yl)-6-(trifluoromethyl)pyrimidin-4-yl)-3-azabicyclo[3.1.0]hexan-6-yl)methanesulfinate;

((1R,5S,6S)-3-(5-methyl-2-((S)-2-methylazetidin-1-yl)-6-(trifluoromethyl)pyrimidin-4-yl)-3-azabicyclo[3.1.0]hexan-6-yl)methanesulfinic acid;

Sodium((1R,5S,6S)-3-(2-((S)-2-methylazetidin-1-yl)-8,8-difluoro-5,6,7,8-tetrahydroquinazolin-4-yl)-3-azabicyclo[3.1.0]hexan-6-yl)methanesulfinate; and

((1R,5S,6S)-3-(2-((S)-2-methylazetidin-1-yl)-8,8-difluoro-5,6,7,8-tetrahydroquinazolin-4-yl)-3-azabicyclo[3.1.0]hexan-6-yl)methanesulfinic acid.

Compounds of Formulae A, A1, B, B1, B2, B3, C and C1 as defined herein are useful as KHK inhibitors but are also believed to have an improved toxicity profile due to a reduced propensity for forming toxic metabolites as compared to corresponding compounds in which the sulfinic acid or sulfonic acid component is replaced with a carboxylic acid component. The compounds or composition of the invention may therefore be used in medicine.

In particular, a compound of the present invention has a different metabolic profile compared to a corresponding carboxylic acid compound (i.e., a compound having a —COOH or —COO⁻ group replacing the mandatory —S(O)OH, —S(O)O—, —S(O)₂OH or —S(O)₂O⁻ group in the compound of Formula A, A1, B, B1, B2, B3, C or C1). These corresponding carboxylic acid compound are typically metabolised to the acyl-glucuronide and such metabolism can give rise to reactive metabolites that cause liver toxicity and drug induced liver injury (Shipkova M, Armstrong V W, Oellerich M, and Wieland E (2003) Acyl glucuronide drug metabolites: Toxicological and analytical implications. Ther Drug Monit 25: 1-16: Regan 5, Maggs J, Hammond T, Lambert C, Williams D and Park B K (2010) Acyl glucuronides: the good, the bad and the ugly. Biopharm Drug Dispos 31: 367-395; Shipkova M, Armstrong V W, Oellerich M, and Wieland E (2003) Acyl glucuronide drug metabolites: Toxicological and analytical implications. Thar Drug Monit 25: 1-16).

For example, the compounds of the present invention may only be metabolised by oxidative pathways, such as Cyp oxidation, and minimise the formation of acyl glucuronide-like metabolites compared to a corresponding carboxylic acid compound.

The compounds of the invention therefore break down in-vivo via a different metabolic pathway than the corresponding carboxylic acid compounds with the result that the compound of the invention have unexpected beneficial liver safety effects and/or improved liver safety and/or improved efficacy compared to corresponding carboxylic acid compounds.

For example, the compounds of the present invention may be more efficacious than, be less toxic than, be longer acting than, be more potent than, produce fewer side effects than, be more easily absorbed than, have a better pharmacokinetic profile (e.g., higher oral bioavailability and/or lower clearance) than, and/or have other useful pharmacological, physical or chemical properties than a compound known in the prior art. Such effects may be evaluated clinically, objectively and/or subjectively by a health care professional, a treatment subject or an observer.

Therefore, in some embodiments, a compound of the present invention may have a different distribution profile when orally dosed in-vivo, such as increased exposure in the liver versus plasma, compared to a corresponding carboxylic acid compound.

In some embodiments, the compounds of the present invention as defined above are metabolites (i.e, having undergone metabolism or biotransformation in the subject).

For example, a compound of the present invention may be a sulfinic acid metabolite, which may be a corresponding sulfonic acid of the compound (e.g., a compound having a —S(O)₂OH or —S(O)₂O⁻ group replacing a —S(O)OH or —S(O)O⁻ group in the compound) or a corresponding sulfinate ester of the compound (e.g., a compound having a —S(O)O(C₁₋₆ alkyl) group replacing a —S(O)OH or —S(O)O⁻ group in the compound).

In other embodiments, the compounds of the present invention as defined above may be sulfinic acid (or its corresponding sulfinate salt) compounds or sulfonic acid (or its corresponding sulfonate salt) compounds. In a preferred aspect, the compounds of the invention are sulfinic acid (or its corresponding sulfinate salt) compounds, i.e. compounds containing -(L)ₘ-S(O)OH or -L-(CH₂)ₙS(O)OH.

In a further embodiment of the invention, the compounds of the present invention are provided in the form of a sodium salt, for example a sodium sulfinate salt.

The reaction schemes described below are intended to provide a general description of the methodology employed in the preparation of the compounds of the present invention. The examples provided herein are offered to illustrate but not limit the compounds of the present invention, as well as the preparation of such compounds and intermediates.

All starting materials, building blocks, reagents, acids, bases, dehydrating agents, solvents and catalysts utilized to synthesize the compounds of the present invention are either commercially available or can be routinely prepared by procedures described in the literature, for example, Houben-Weyl “Science of Synthesis” volumes 1-48, Georg Thieme Verlag, and subsequent versions thereof.

A reaction may be carried out in the presence of a suitable solvent or diluent or of mixture thereof in a manner known to those skilled in the art of organic synthesis. A reaction may also be carried out, if needed, in the presence of an acid or a base, with cooling or heating, for example in a temperature range from about −30° C. to about 150° C. In some embodiments, a reaction is carried out in a temperature range from about 0° C. to about 100° C., and more particularly, in a temperature range from room temperature to about 80° C., in an open or closed reaction vessel and/or in the atmosphere of an inert gas, for example nitrogen.

An illustration of the preparation of compounds of the present invention is shown below. Unless otherwise indicated in the schemes, the variables have the same meaning as described above.

In the Formulas of general synthesis schemes depicted below L₁ is

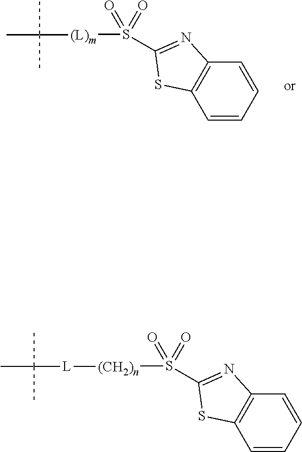

Wherein the variants L, m and n are as defined in the claims.

L₂ is independently selected from —C₁₋₃alkyl and —OH, wherein —C₁₋₃alkyl is substituted with

0 to 3 halogen atoms.

P is 1, 2, 3 or 4

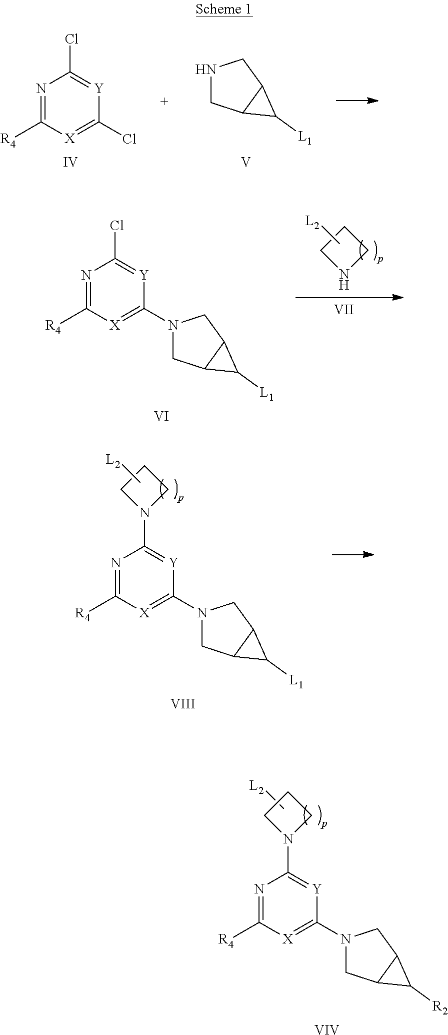

In Scheme 1 shown above, the variants R_{2(a-d)}, R_{4(a-d)}, X_{(a-d)} and Y_{(a-d)} are as defined above and in the claims. Heterocycles of Formula IV are reacted with amine V to give the product of Formula VI which is reacted with a second amine VII to give the compound of Formula VIII. This can be achieved using standard procedures for the S_{N}Ar amination of halogenated heterocycles in the presence of a base (such as potassium carbonate or DIPEA). An example would be TEA in CCM at room temperature or heated at 60° C. in MeCN with TEA. Alternatively, standard conditions for Buchwald-Hartwig cross coupling for the amination of aromatic compounds may be employed, using a palladium catalyst (such as (DPPF)PdCl₂, Pd₂(dba)₃, base (such as NaO^{t}Bu, KOH) and ligand (such as BINAP, MePhos) in a suitable solvent. The compounds of Formula VIII can then be converted to the desired sulfinic acids using, for example, NaOMe in methanol.

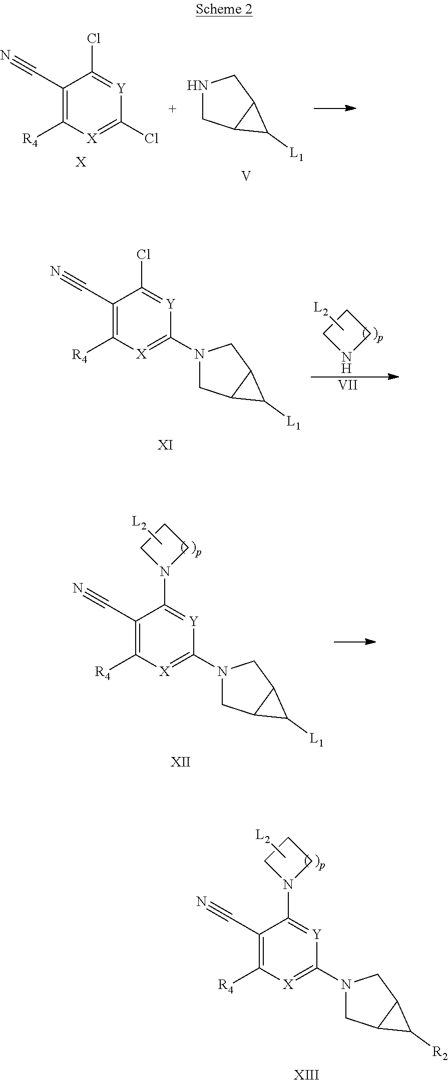

In Scheme 2 shown above, the variants R_{2(a-d)}, R_{4(a-c)}, X_{(a-d)} and Y_{(a-d)} are as defined in the claims. Heterocycles of Formula X are reacted with amine V to give the product of Formula XI which is reacted with a second amine VII to give the compound of Formula XII. This can be achieved using standard procedures for the S_{N}Ar amination of halogenated heterocycles in the presence of a base (such as potassium carbonate or DIPEA) An example would be TEA in DCM at room temperature or heated at 60° C. with TEA in MeCN. Alternatively, standard conditions for Buchwald-Hartwig cross coupling for the amination of aromatic compounds may be employed, using a palladium catalyst (such as (DPPF)PdCl₂, Pd₂(dba)₃, base (such as NaO^{t}Bu, KOH) and ligand (such as BINAP,

MePhos) in a suitable solvent. The compounds of Formula XII can then be converted to the desired sulfinic acids using, for example, NaOMe in methanol.

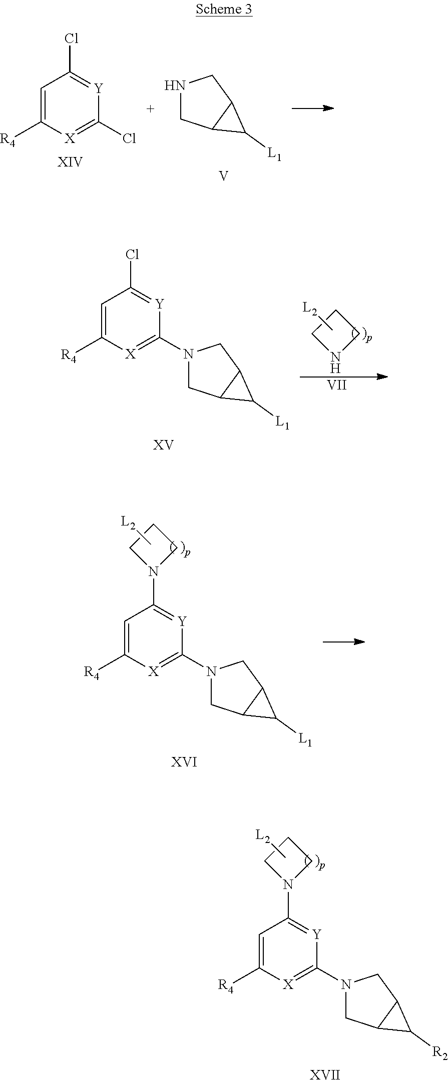

In Scheme 3 shown above, the variants R_{2(a-d)}, R_{4(a-d)}, X_{(a-d)} and Y_{(a-d)} are as defined in the claims. Heterocycles of Formula XIV are reacted with amine V to give the product of Formula XV which is reacted with a second amine XVI to give the compound of Formula XVII. This can be achieved using standard procedures for the S_{N}Ar amination of halogenated heterocycles in the presence of a base (such as potassium carbonate or DIPEA). An example would be TEA in DCM at room temperature or heated at 60° C. with TEA in MeCN. Alternatively, standard conditions for Buchwald-Hartwig cross coupling for the amination of aromatic compounds may be employed, using a palladium catalyst (such as (DPPF)PdCl₂, Pd₂(dba)₃, base (such as NaO^{t}Bu, KOH) and ligand (such as BINAP, MePhos) in a suitable solvent. The compounds of Formula XII can then be converted to the desired sulfinic acids using, for example, NaOMe in methanol.

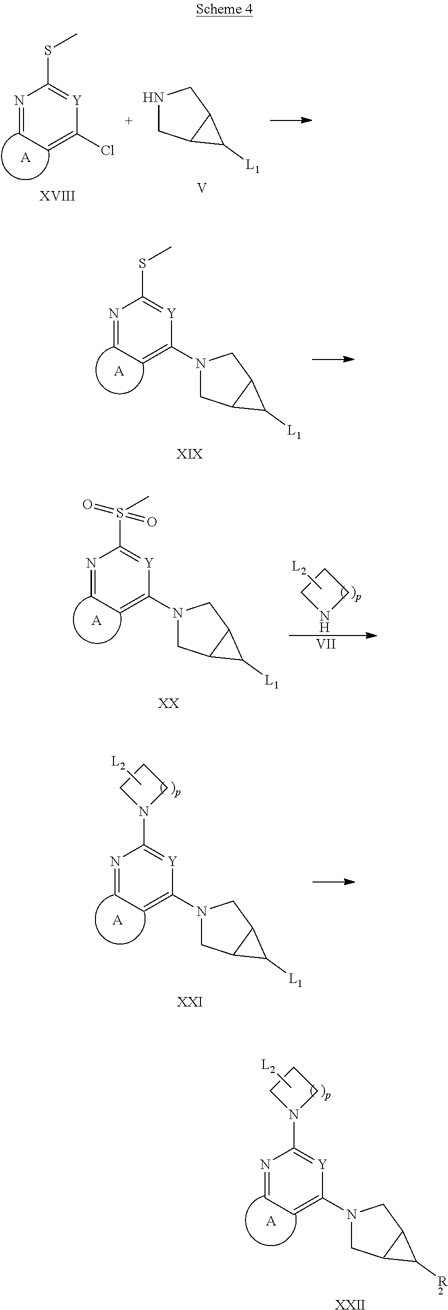

In Scheme 4 shown above, the variants R_{2(a-d)}, Y_{(a-d)} and Ring A, are as defined above and in the claims. Heterocycles of Formula XVIII, prepared for example as described in WO2020156445, are reacted with amine V to give the product of Formula XIX which can be oxidised to the arylsulfone XX, such as with m-CPBA in an inert solvent e.g. DCM. Compounds of formula XX are then reacted with a second amine VII to give the compound of Formula XXI. This can be achieved using standard procedures for the S_{N}Ar amination of heterocycles in the presence of a base (such as potassium carbonate or DIPEA) in a suitable solvent. An example would be DIPEA in NMP at heated at 160° C. The compounds of Formula XXI can then be converted to the desired sulfinic acids using, for example, NaOMe in methanol.

Provided according to the present invention is a composition (i.e. a pharmaceutical or veterinary composition) comprising a compound of the present invention (i.e, a compound of Formula A, A1, B, B1, B2, B3, C or C1 or an enantiomer, stereoisomer, tautomer, solvate, hydrate, prodrug, amino acid conjugate, metabolite, or pharmaceutically acceptable salt thereof). The compositions comprise an excipient, diluent and/or carrier as required. For example, in one embodiment, the invention relates to a pharmaceutical composition comprising a compound of the present invention (e.g, a compound of Formula A, A1, B, B1, B2, B3, C or C1 or an enantiomer, stereoisomer, tautomer, solvate, hydrate, prodrug, amino acid conjugate, metabolite, or pharmaceutically acceptable salt thereof) and a pharmaceutically acceptable excipient, diluent and/or carrier.

As used herein, the term “pharmaceutically acceptable excipient, diluent and/or carrier” includes any and all solvents, dispersion media, coatings, surfactants, antioxidants, preservatives (e.g., antibacterial agents, antifungal agents), isotonic agents, absorption is delaying agents, salts, preservatives, drug stabilizers, binders, excipients, disintegration agents, lubricants, sweetening agents, flavoring agents, dyes, and the like and combinations thereof, that are suitable for use in a pharmaceutical product. Such excipients, diluents and/or carriers include those which would be known to those skilled in the art (see, for example, Remington's Pharmaceutical Sciences, 18th Ed. Mack Printing Company, 1990, pp. 1289-1329). Except insofar as any conventional excipient, diluent and/or carrier is incompatible with the active ingredient (e.g., a compound of the present invention), its use in the therapeutic and/or pharmaceutical compositions is contemplated

Compounds of the invention are indicated as pharmaceuticals. Therefore, according to a further aspect of the invention, there is provided a compound of the invention (i.e. a compound of Formula A, A1, B, B1, B2, B3, C or C1 or an enantiomer, stereoisomer, tautomer, solvate, hydrate, prodrug, amino acid conjugate, metabolite, or pharmaceutically acceptable salt thereof), or a pharmaceutical (or veterinary) composition of the invention for use as a pharmaceutical (e.g, for use in medicine).

According to aspects and embodiments of the present invention, the compounds of the invention or compositions of the invention may be administered to patients or subjects in need thereof.

As used herein, the term “patient” and “subject” refer to an animal. Typically, the animal is a mammal. A “patient” or “subject” also refers to, for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

Therefore, the present invention provides a method of inhibiting KHK, the method comprising administering to a subject in need thereof an effective amount of a compound of the invention or a pharmaceutical composition of the invention. Said methods are hereinafter referred to as “methods of the invention”.

The present invention also provides a compound of the invention or a pharmaceutical composition of the invention for use in inhibiting KHK. The present invention also provides the use of a compound of the invention or a pharmaceutical composition of the invention in the manufacture of a medicament for inhibiting KHK.

In some embodiments, a KHK plays a role in a disease or disorder, i.e. KHK is involved in a pathway, mechanism, or action associated with the disease and/or disorder such as, e.g. transcriptional regulation of glucose and lipid metabolism.

The present invention therefore also provides a method of treating and/or preventing a disease or disorder in which KHK plays a role, the method comprising administering to a subject in need thereof an effective amount of a compound of the invention or a pharmaceutical composition of the invention.

The present invention also provides a compound of the invention or a pharmaceutical composition of the invention for use treating and/or preventing a disease or disorder in which KHK plays a role. The present invention also provides the use of a compound of the invention or a pharmaceutical composition of the invention in the manufacture of a medicament for treating and/or preventing a disease or disorder in which KHK plays a role.

In some embodiments, the disease or disorder is selected from the group consisting of diabetes (T1D and/or T2D), idiopathic T1D (Type 1b), latent autoimmune diabetes in adults (LADA), early-onset T2D (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, obesity, eating disorders, excessive sugar craving, dyslipidemia (including hyperlipidemia, hypertriglyceridemia, increased total cholesterol, high LDL cholesterol, and low HDL cholesterol), hyperinsulinemia, NAFLD (including related diseases such as steatosis, NASH, fibrosis, cirrhosis, and hepatocellular carcinoma), HFI, coronary artery disease, peripheral vascular disease, hypertension, endothelial dysfunction, impaired vascular compliance, congestive heart failure, myocardial infarction (e.g. necrosis and apoptosis), stroke, hemorrhagic stroke, ischemic stroke, pulmonary hypertension, restenosis after angioplasty, intermittent claudication, post-prandial lipemia, metabolic acidosis, ketosis, arthritis, osteoporosis, left ventricular hypertrophy, peripheral arterial disease, macular degeneration, cataract, glomerulosclerosis, chronic renal failure, metabolic syndrome, syndrome X, premenstrual lo syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, foot ulcerations, ulcerative colitis, hyper apo B lipoproteinemia, Alzheimer's Disease, schizophrenia, impaired cognition, inflammatory bowel disease, ulcerative colitis, Crohn's is disease, and irritable bowel syndrome.

The term “therapeutically effective amount” refers to an amount of a compound of the present invention (i.e., a compound of Formula A, A1, B, B1, B2, B3, C or C1) and/or composition of the invention that is sufficient to achieve or elicit a therapeutically useful response or a stated effect in a subject. Accordingly, a therapeutically effective amount of a compound of Formula A (or similarly Formula A1, B, B1, B2, B3, C or C1) used for the treatment of a condition mediated by KHK can be an amount sufficient for the treatment of the condition mediated by KHK.

The terms “treat”, “treating”, “treatment” or and grammatical variations thereof in reference to a disease, or condition refer to any type of treatment that imparts a benefit to a subject and may mean that the severity of the subject's condition is reduced, at least partially improved or ameliorated and/or that some alleviation, mitigation or decrease in at least one clinical symptom associated with a disease, disorder, or condition is achieved and/or there is a delay in the progression of the symptom. In some embodiments, the severity of a symptom associated with a disease, disorder, or condition mediated by KHK may be reduced in a subject compared to the severity of the symptom in the absence of a method of the present invention. In some embodiments, “treat”, “treating”, “treatment” or and grammatical variations thereof in reference to a disease or disorder refer to ameliorating the disease or disorder (i.e., slowing or arresting or reducing the development of the disease or disorder or at least one clinical symptom thereof). In some embodiments, “treat”, “treating” or “treatment of” and grammatical variations thereof in reference to a disease or disorder refer to alleviating or ameliorating at least one physical parameter including those which may not be discernible by the subject. In some embodiments, “treat”, “treating” or “treatment of” and grammatical variations thereof in reference to a disease or disorder refer to modulating the disease or disorder. either physically (e.g., stabilization of a discernible symptom), physiologically (e.g., stabilization of a physical parameter), or both.

In some embodiments, a compound of the present invention and/or composition of the invention may be administered to a subject in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with a disease or disorder (e.g., a disease, disorder, or condition mediated by KHK) and/or a reduction in the severity of the onset of symptom associated with a disease or disorder (e.g., a disease, disorder, or condition mediated by KHK) relative to what would occur in the absence of a method of the present invention. The prevention can be complete. e.g.. the total absence of the symptom. The prevention can also be partial, such that the occurrence of the symptom in the subject and/or the severity of onset is less than what would occur in the absence of a method of the present invention.

In some embodiments, a compound of the present invention and/or composition of the invention may be administered in a prevention effective amount. A “prevention effective” amount as used herein is an amount that is sufficient to prevent (as defined herein) a symptom associated with a disease or disorder (e.g., a disease, disorder, or condition mediated by KHK) in a subject. Those skilled in the art will appreciate that the level of prevention need not be complete, as long as some benefit is provided to the subject. In some embodiments, a prevention effective amount may be achieved by administering a composition of the present invention.

The terms “administer”, “administering”, “administration” and grammatical variations thereof as used herein refer to directly administering to a subject a compound of the present invention (or a pharmaceutically acceptable salt, etc., thereof) and/or a composition of the present invention. In some embodiments, a compound and/or composition of the present invention is administered to the subject in an amount that can form an equivalent amount of the active compound within the subject's body.

A compound of the present invention and/or composition of the present invention can be administered in a therapeutically effective amount to treat and/or prevent a disease or disorder and/or to prevent the development thereof in a subject. Administration of a compound of the present invention can be accomplished via any mode of administration for therapeutic agents such as, for example oral, rectal, topical, and/or parenteral administration may be employed. In some embodiments, a compound of the present invention is administered orally.

Depending on the intended mode of administration, a compound of the present invention and/or composition of the present invention can be in a dosage form known to those skilled is in the pharmaceutical practices, such as, for example, injectables, tablets, suppositories, pills, time-release capsules, emulsions, syrups, powders, liquids, suspensions, and/or the like.

Typical pharmaceutical compositions include, but are not limited to, tablets, pills, powders or gelatin capsules comprising the active ingredient (e.g., a compound of the present invention) and a pharmaceutically acceptable carrier such as for example:

a) a diluent, e.g., purified water, corn oil, olive oil, sunflower oil, fish oils, such as EPA or DHA or their esters or triglycerides or mixtures thereof, lactose, dextrose, sucrose, mannitol, sorbitol, cellulose and/or glycine;

b) a lubricant, e.g., silica, talcum, stearic acid its magnesium or calcium salt and/or polyethylene glycol; for tablets also;

c) a binder, e.g., magnesium aluminum silicate, starch paste, gelatin, tragacanth, methylcellulose, sodium carboxymethylcellulose, natural and synthetic gums such as acacia tragacanth or sodium alginate, waxes and/or polyvinylpyrrolidone, if desired;

d) a disintegrant, e.g., starches, agar, algic acid or its sodium salt, and/or effervescent mixtures;

e) absorbent, colorant, flavorant and/or sweetener;

f) an emulsifier or dispersing agent, e.g. Labrasol, HPMC, labrafil, peceol, capmul, vitamin E TGPS and/or other acceptable emulsifier; and/or

g) an agent that enhances absorption of the compound such as cyclodextrin, hydroxypropyl-cyclodextrin, PEG400, and/or PEG200.

Liquid, particularly injectable, compositions can, for example, be prepared by dissolution, dispersion, etc. For example, a compound of the present invention is dissolved in or mixed with a pharmaceutically acceptable solvent such as, for example, water, saline, aqueous dextrose, glycerol, ethanol, and/or the like, to thereby form an injectable isotonic solution or suspension. Said composition may be sterilized and/or contain adjuvants, such as preserving, stabilizing wetting or emulsifying agents, solution promoters, salts for regulating osmotic pressure and/or buffers.

A compound of the present invention and/or composition of the present invention may also be formulated as a suppository that can be prepared from fatty emulsions or suspensions; using polyalkylene glycols such as propylene glycol, as the carrier.

A compound of the present invention and/or composition of the present invention may also be delivered by the use of monoclonal antibodies as individual carriers to which the compound is coupled.

A compound of the present invention and/or composition of the present invention may be coupled with a soluble polymer as a targetable drug carrier. Such polymers can include, but are not limited to. polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, a compound of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphiphillic block copolymers of hydrogels. In one embodiment disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection. In addition, they may also contain other therapeutically valuable substances. Said compositions may be prepared according to conventional mixing, granulating and/or coating methods, respectively, and contain about 0.1-75%, or contain about 1-50%, of the active ingredient.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the compounds of this invention, excipients such as lactose, talc, silicic acid, aluminum hydroxide, calcium silicates and polyimide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing a compound of the present invention in the proper medium. Absorption enhancers can also be used to increase the flux of the compound across the skin. The rate can be controlled by either providing a rate controlling membrane or by dispersing the compound in a polymer matrix or gel.

Compositions of the present invention can be prepared according to conventional mixing, granulating and/or coating methods, respectively, and the present pharmaceutical compositions can contain from about 0.1% to about 99% of compound by weight or volume.

The present invention further provides pharmaceutical compositions and dosage forms that comprise one or more agents that reduce the rate by which a compound of the present invention as an active ingredient will decompose. Such agents, which are referred to herein as “stabilizers” include, but are not limited to, antioxidants such as ascorbic acid, pH buffers, and/or salt buffers, etc.

The dosage regimen utilizing a compound of the present invention may be selected in accordance with a variety of factors including type, species, age, weight, sex and/or medical condition of the subject; the severity of the condition to be treated; the route of administration; the renal or hepatic function of the subject; and the particular disclosed compound employed. A physician, clinician or veterinarian of ordinary skill can readily determine the effective amount of each of the active ingredients necessary to prevent, treat or inhibit the progress of the disorder or disease.

Effective dosage amounts of a compound of the present invention, when used for the indicated effects, range from about 0.5 mg to about 5000 mg of the compound as needed to treat the condition.

In some embodiments, a compound of the present invention is an isotopically labelled compound. An “isotopically labelled compound” as used herein refers to a compound in which at least one atomic position is enriched in a specific isotope of the designated element to a level which is significantly greater than the natural abundance of that isotope. For example, one or more hydrogen atom positions in a compound can be enriched with deuterium to a level that is significantly greater than the natural abundance of deuterium, for example, enrichment to a level of at least 1%, preferably at least 20% or at least 50%. Such a deuterated compound may, for example, be metabolized more slowly than its non-deuterated analogue, and therefore exhibit a longer half-life when administered to a subject (Annual Reports In Medicinal Chemistry, Vol. 26, 2011, Chapter 24—Deuterium is in Drug Discovery and Development, pages 403-417). Such compounds can be synthesized using methods known in the art, for example, by employing deuterated starting materials. Unless stated to the contrary, isotopically labelled compounds are pharmaceutically acceptable.

The invention will now be described by reference to the following, non-limiting, figure and examples.

### BRIEF DESCRIPTION OF THE FIGURE

FIG. 1: Graph showing the change in fructose levels from baseline in the control and Example 2 and 6 treated animals in the OFTT study. The increase of fructose plasma levels on dosing with a test compound versus the control group indicate that KHK is being inhibited, thus reducing the rate of elimination of fructose from the plasma.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

In the case of a discrepancy between the name of a compound and the accompanying structure presented herein, the structure provided should prevail.

Abbreviations

aq, Aqueous

br broad

d doublet

DCM dichloromethane

dd doublet of doublets

DIPEA N,N-Diisopropylethylarnine

DMSO dimethylsulfoxide

EtOAc ethyl acetate

EtOH ethanol

g gram

h hour(s)

H₂ hydrogen

IC₅₀ concentration of half maximal inhibition

KOH Potassium Hydroxide

LCMS liquid chromatography and mass spectrometry

m multiplet

M molar

MeCN acetonitrile

MeOH methanol

MS mass spectrometry

NaHCO₃ sodium hydrogencarbonate

NaOMe sodium methoxide

NH₄Cl ammonium chloride

mg milligram

min(s) minute(s)

ml milliliter

mmol millimole

MTBE methyl tert-butylether

Na₂SO₄ sodium sulfate

NMR nuclear magnetic resonance

O₂ oxygen

pet petroleum

s singlet

sat. saturated

sext sextet

t triplet

tert tertiary

td triplet of doublets

### Reference Example 1—Preparation of 2-(((1R,5S,6r)-3-(2-chloro-6-(trifluoromethyl)pyrimidin-4-yl)-3-azabicyclo[3.1.0]hexan-6-yl)methylthio)benzo[d]thiazole

### (1R,5S,6r)-tert-butyl-6-((methylsulfonvloxy)ethyl)-3-azabicyclo[3.1.0]hexane-3-carboxylate

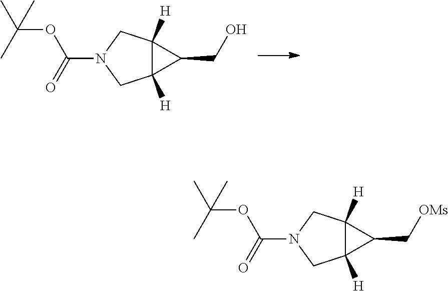 [SMILES: CC(C)(C)OC(=O)N1C[C@@H]2[C@@H](CO)[C@@H]2C1 ; CC(C)(C)OC(=O)N1C[C@@H]2[C@@H](COS(C)(=O)=O)[C@@H]2C1]

A solution of methane sulfonyl chloride (4.4 ml, 56.33 mmol) in dichloromethane (ml) was added to a solution of (1R,5S,6r)-tert-butyl-6-(hydroxymethyl)-3-azabicyclo[3.1.0]hexane-3-carboxylate (10.0 g, 46.9 mmol) and triethylamine (19.6 ml, 140.8 mmol) in dichloromethane (30 ml) at 0° C. After 4 h the mixture was warmed to room temperature and concentrated to dryness to afford the titled compound (13 g, 95%) which was used in the next step without further purification.

### (1R,5S,6r)-tert-butyl6-((benzo[d]thiazol-2-ylthio)methyl)-3-azabicyclo[3.1.0]hexane-3-carboxylate

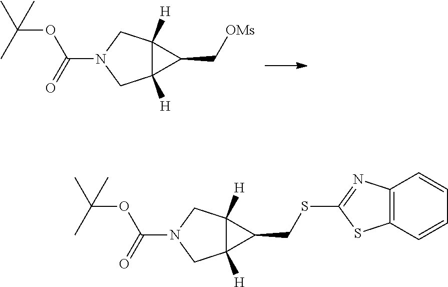 [SMILES: CC(C)(C)OC(=O)N1C[C@@H]2[C@@H](COS(C)(=O)=O)[C@@H]2C1 ; CC(C)(C)OC(=O)N1C[C@@H]2[C@@H](CSc3nc4ccccc4s3)[C@@H]2C1]

Potassium carbonate (18.5 g, 134 mmol) was added to a stirred suspension of (1R,5S,6r)-tert-butyl-6-((methylsulfonyloxy)methyl)-3-azabicyclo[3.1.0]hexane-3-carboxylate (13 g, 44.6 mmol) and benzo[d]thiazole-2(3H)-thione (7.5 g. 44.6 mmol) in DMF (50 ml) at 65° C. After 16 h the reaction mixture was allowed to cool to room temperature and partitioned between ethyl acetate and water. The separated organic layer was washed with brine, dried over Na₂SO₄, filtered and concentrated under reduced pressure. The residue was purified by column chromatography using 5% ethyl acetate in pet ether as eluent to afford the titled compound as a solid (7 g, 43%). ¹H NMR (400 MHz, CDCl₃): δ 7.88-7.84, (m, 1H), 7.77-7.74 (m, 1H), 7.44-7.39 (m, 1H), 7.32-7.27 (m, 1H), 3.65-3.52 (m, 2H .41-3.31 (m, 4H), 1.62-1.55 (m, 2H), 1.42 (s, 9H), 1.15-1.05 (m, 1H).

### 2-((1R,5S,6r)-3-azabicyclo[3.1.0]hexan-6-ylmethylthio)benzo[d]thiazole hydrochloride

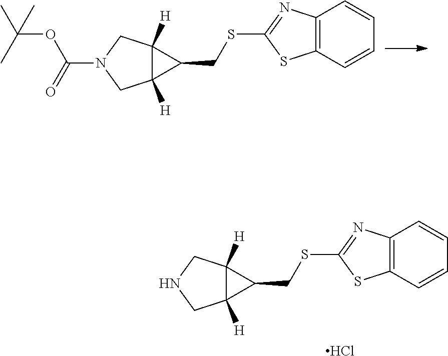 [SMILES: CC(C)(C)OC(=O)N1C[C@@H]2[C@@H](CSc3nc4ccccc4s3)[C@@H]2C1 ; c1ccc2sc(SC[C@H]3[C@@H]4CNC[C@@H]43)nc2c1 ; Cl]

(1R,5S,6r)-tert-butyl6-((benzo[d]thiazol-2-ylthio)methyl)-3-azabicyclo[3.1.0]hexane-3-carboxylate (7 g, 19.33 mol) was dissolved in 4M Dioxane HCl (70 ml) and the solution stirred at room temperature for 3 h. The solvent was removed under reduced pressure to give the titled as a solid (6 g, 100%). LC-MS: 1.81mins, [MSH]⁺ 263

### 2-(((1R,5S,6r)-3-(2-chloro-6-(trifluoromethyl)pyrimidin-4-yl)-3-azabicyclo[3.1.0]hexan-6-yl)methylthio)benzordlthiazole

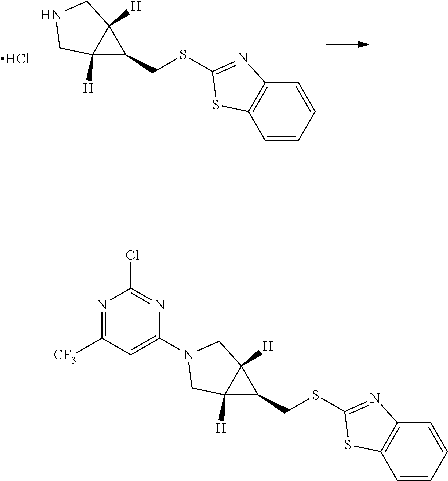 [SMILES: Cl ; c1ccc2sc(SC[C@H]3[C@@H]4CNC[C@@H]43)nc2c1 ; FC(F)(F)c1cc(N2C[C@@H]3[C@@H](CSc4nc5ccccc5s4)[C@@H]3C2)nc(Cl)n1]

A solution of 2,4-dichloro-6-(trifluoromethyl)pyrimidine (4.0 g, 18.43 mmol) in DCM (10 ml) and then DIPEA (1.46 ml) were added to a solution of 2-((1R,5S,6r)-3-azabicyclo[3.1.0]hexan-6-ylmethylthio)benzo[d]thiazole hydrochloride (5 g, 16.83 mmol) in DCM (50 ml) at 0° C. The mixture was then allowed to warm to room temperature. After 4 h the reaction mixture was diluted with DCM, washed with water and the DCM layer dried over Na₂SO4, filtered and concentrated under reduced pressure. The residue was purified by chromatography on silica gel (eluent: pet ether/ethyl acetate=9/1) to give the titled compound as a solid (4.0 g, Yield: 54%). ¹H NMR (400 MHz, d⁶-DMSO): 7.99 (dd, 1H), 7.84 (dd, 1H), 7.45 (m, 1H), 7.35 (td, 1H), 6.91, (s, 1H), 3.82-3.73 (m, 2H), 3.61-3.52 (m, 2H), 3.50-3.44 (m, 2H), 3.41-3.35 (m, 2H), 1.95-1.85 (m, 2H), 1.18-1.12 (m, 1H).

### Example 2—Preparation of sodium((1R,5S,6S)-3-(2-((S)-2-methylazetidin-1-yl)-6-(trifluoromethyl)pyrimidin-4-yl)-3-azabicyclo[3.1.0]hexan-6-yl)methanesulfinate

### (S)-1-benzhydryl-2-methylazetidine

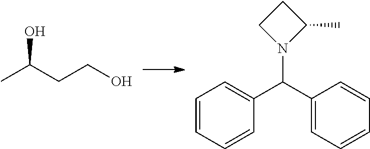

Trifluoromethanesulfonic anhydride (5.8 ml, 14.587 mmol) was added dropwise to a solution of (R)-1.3-butanediol (5 g. 55.5 mmol) and DIPEA (50 ml) in MeCN (40 ml) at −30° C. After 20 min a solution diphenylmethanamine (9.8 ml) in MeCN (10 ml) was added and the resulting mixture was stirred for 20 min at −30° C. The reaction mixture was then allowed to warm to room temperature, stirred for a further 30 mins before being heated at 45° C. On completion of the reaction, the mixture was poured into water and toluene, The organic layer was washed with water, brine solution, dried over sodium sulphate, filtered and concentrated. The crude product was purified by silica gel column chromatography eluting with 0-20% pet ether and ethyl acetate, to give the titled compound (8.0 g, 61% yield) as an oil. LC-MS: 3.59 mins, [M+H]⁺238

### (2S)-1-benzhydryl-2-methylazetidinium((1R,4R)-7,7-dimethyl-2-oxobicyclo[2.2.1]heptan-1-yl)methanesulfonate

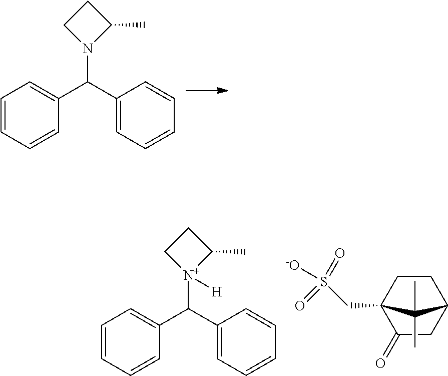

A solution of (S)-1-benzhydryl-2-methylazetidine (8.0 g, 33.75 mmol), and ((1R,4R)-7,7-dimethyl-2-oxobicyclo[2.2.1]heptan-1-yl)methanesulfonate (7.9 g, 34.05 mol) in methanol (32 ml) was stirred at room temperature, After 12 h reaction the mixture was concentrated under reduced pressure and the crude product dissolved in DCM. EtOAc was added, the mixture stirred for a further 1 h at room temperature before being filtered and the solid washed with EtOAc and dried, The obtained solid was again dissolved in DCM, EtOAc added and the mixture stirred at room temperature for 4 h before being filtered, washed with EtOAc and dried to give the titled compound (10.0 g, 63% yield) as a solid. LC-MS: 2.68 mins, [M+H]⁺238

### (S)-2-methylazetidinium((1R,4R)-7,7-dimethyl-2-oxobicyclo[2.2.1]heptan-1-yl)methanesulfonate

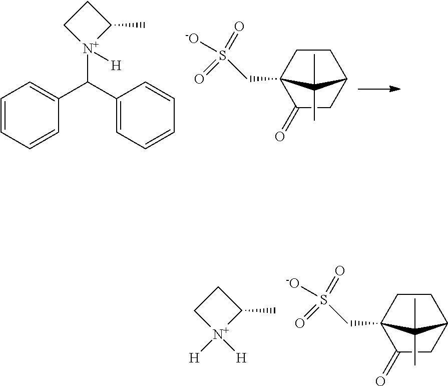

20% Pd(OH)/C (0.19 g) was added to a stirred suspension of (2S)-1-benzhydryl-2-methylazetidinium ((1R,4R)-7,7-dimethyl-2-oxobicyclo[2.2.1]heptan-1-yl)methanesulfonate (3.0 g, 6.39 mmol) in methanol (15 ml) in a hydrogenation vessel. The reaction mixture was degassed, filled with H₂ (60 psi) and stirred for 16 h at room temperature. After completion of the reaction, the mixture was filtered through celite, washed with methanol and the filtrate concentrated under reduced pressure. The crude product was then added to 1:1 EtOAc/MTBE (30 ml) and heated at 60° C. for 1 h. The mixture was then cooled to room temperature, stirred for 1 h and the solid isolated by filtration. The solid was added to MTBE, stirred for 2 h at room temperature, filtered, washed with MTBE and dried to give titled compound as a solid (1.5 g, 77% yield). ¹H NMR (400 MHz, MeOD): 4.60 (sext, 1H), 4.08-3.98 (m, 1H), 3.89 (td, 1H), 3.22-3.27 (m, 1H), 2.77 (d, 1H), 2.70-2.55 (m, 2H), 2.49-2.25 (m, 2H), 2.11-1.98 (m, 2H), 1.89 (d, 1H), 1.68-1.57 (m, 1H), 1.54 (d, 3H), 1.48-1.37 (m, 1H), 1.11 (s, 3H), 0.86 (5, 3H).

### 2-(((1R,5S,6S)-3-(2-((S)-2-methylazetidin-1-yl)-6-(trifluoromethyl)pyrimidin-4-yl)-3-azabicyclo[3.1.0]hexan-6-yl)methylthio)benzo[d]thiazole

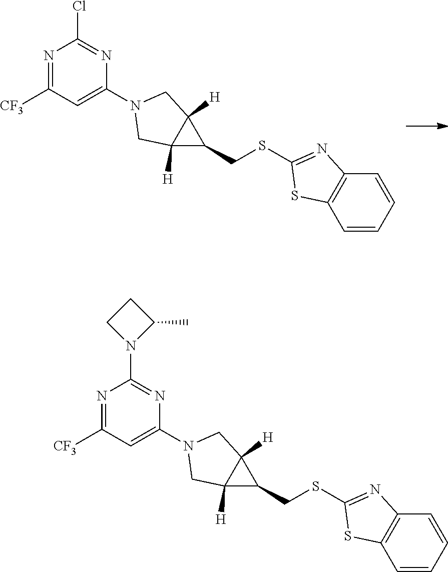 [SMILES: FC(F)(F)c1cc(N2C[C@@H]3[C@@H](CSc4nc5ccccc5s4)[C@@H]3C2)nc(Cl)n1 ; C[C@H]1CCN1c1nc(N2C[C@@H]3[C@@H](CSc4nc5ccccc5s4)[C@@H]3C2)cc(C(F)(F)F)n1]

DIPEA (1.2 ml) was added to a mixture of 2-(((1 R,5S,6r)-3-(2-chloro-6-(trifluoromethyl)pyrimidin-4-yl)-3-azabicyclo[3.1.0]hexan-6-yl)methylthio)benzo[d]thiazole (1.0 g, 2.26 mmol) and (S)-2-methylazetidinium ((1R,4R)-7,7-dimethyl-2-oxobicyclo[2.2.1]heptan-1-yl) methanesulfonate (0.75 g, 2.48 mmol) in MeCN (10 ml). The reaction mixture was heated to 60° C. and after 12 h concentrate under reduced pressure. The crude product was purified by chromatography on silica gel eluting with 9:1 petroleum ether/EtOAc to give the titled compound (1.0 g, Yield: 90%). ¹H NMR (400 MHz, CDCl₃): 7.86 (dd, 1H), 7.75 (dd, 1H), 7.41 (td, 1H), 7.29 (d, 1H), 5.89 (s, 1H), 4.41 (sext, 1H), 4.05-3.88 (m, 3H), 3.60-3.35 (5H), 2.42-2.30 (m, 1H), 1.98-1.88 (1H), 1.76 (bs, 2H), 1.48 (d, 3H), 1.18-1.12 (m, 1H); LC-MS: 5.28 mins, [M+H]⁺478.

### 2-(((1R,5S,6S)-3-(2-((S)-2-methylazetidin-1-0-6-(trifluoromethyl)pyrimidin-4-yl)-3-azabicyclo[3.1.0]hexan-6-yl)methylsulfonyl)benzo[d]thiazole

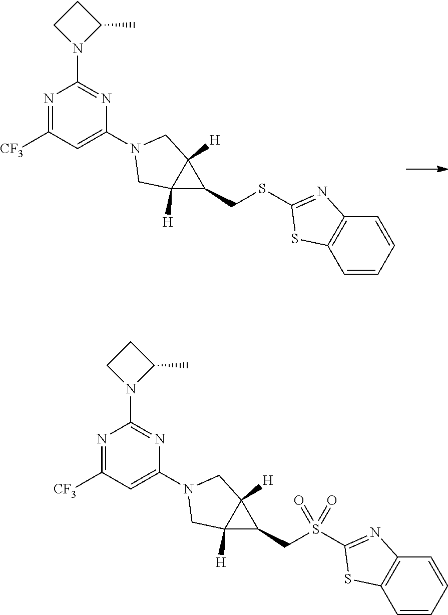 [SMILES: C[C@H]1CCN1c1nc(N2C[C@@H]3[C@@H](CSc4nc5ccccc5s4)[C@@H]3C2)cc(C(F)(F)F)n1 ; C[C@H]1CCN1c1nc(N2C[C@@H]3[C@H](C2)[C@H]3CS(=O)(=O)c2nc3ccccc3s2)cc(C(F)(F)F)n1]

Ammonium molybdate (0.57 g, 0.45 mmol) and then 30% aq. H₂O₂, dropwise, were added to a solution of 2-(((1R ,5S,6S)-3-(2-((S)-2-methylazetidin-1-yl)-6-(trifluoromethyl)pyrimidin-4-yl)-3-azabicyclo[3.1.0]hexan-6-yl)methylthio)benzo[d]thiazole (1.1 g, 2.3 mmol) in EtOH (10 ml) at 0° C. The reaction mixture was then allowed to warm to room temperature. After 5 h, water was added, the solid isolated by filtration and purified by silica gel column chromatography eluting with 20% EtOAc in pet ether to give the titled compound as a solid (0.45 g, Yield: 34%). ¹H NMR (400 MHz, CDCl₃): 7.86 (dd, 1H), 7.75 (dd, 1H), 7.41 (td, 1H), 7.29 (d, 1H), 5,87 (s, 1H), 4.41 (sext, 1H), 4.07-3.85 (m, 3H), 3.62-3.32 (5H), 2.42-2.30 (m, 1H), 1.98-1.88 (1H), 1,71 (bs, 2H), 1.47 (d, 3H), 1.08-0.99 (m, 1H); LC-MS: 5.28 mins, [M+H]⁺510.

### Sodium((1R,5S,6S)-3-(2-((S)-2-methylazetidin-1-yl)-6-(trifluoromethyl)pyrimidin-4-yl)-3-azabicyolo[3.1.0]hexan-6-yl)methanesulfinate

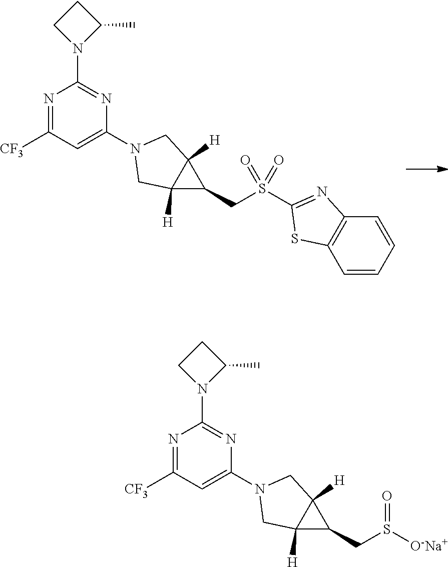

0.5M sodium methoxide solution in methanol (1.14 ml, 0.785 mmol) added slowly dropwise to a solution of 2-(((1R,5S,6S)-3-(2-((S)-2-methylazetidin-1-yl)(trifluoromethyl)pyrimidin-4-yl)-3-azabicyclo[3.1.0]hexan-6-yl)methylsulfonyl)benzo[d]thiazole (0.4 g, 0.785 mmol), in MeOH (3 ml) at 0° C. The reaction mixture was allowed to warm to room temperature and stirred for a further 3 h before being concentrated under reduced pressure, The crude product was washed with n-pentane and dried to give the titled compound as white solid (0.14 g, Yield: 44%). ¹H NMR (400 MHz, d⁶-DMSO): 6.11 (s, 1H), 4.41-4.29 (m, 1H), 3.92-3.73 (m. 3H), 3.59-3.50 (1H), 3.45-3.33 (m, 2H), 2.42-2.30 (m, 1H), 1.94-1.82 (1H), 1.82-1-70 (m, 2H), 1.55 (bs, 2H), 1.42 (d, 3H), 0.64-0.55 (m, 1H); LC-MS: 3.94 mins, [M+H]⁺377.

### Example 3—Preparation of Sodium((1R,5S,6S)-3-(2-((2S,3R)-3-hydroxy-2-methylazetidin-1-yl)-6-(trifluoromethyl)pyrimidin-4-yl)-3-azabicyclo[3.1.0]hexan-6-yl)methanesuifinate

### (2S,3R)-1-benzhydryl-2-methylazetidin-3-ol

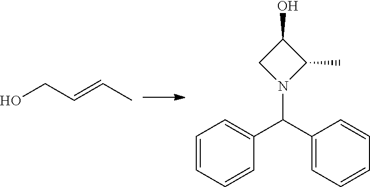

Bromine (7.5 ml) was added to a solution of 2-buten-1-ol (15 g, 208 mmol) in DCM (160 ml) at −78° C. The reaction mixture was then allowed to slowly warm to room temperature, diluted with DOM (100 ml), washed with sat. sodium thiosulphate solution, dried and concentrated under reduced pressure. The crude product was then dissolved in ether (120 ml), cooled to 0° C. and a solution of KOH (14 g) in water (100 ml) slowly added dropwise. The reaction mixture was then allowed to warm to room temperature, stirred for 12 h, heated at 50° C. for 4 h, cooled again to room temperature and stirred for a further 12 h.

The reaction was diluted with EtOAc, washed with water, dried over sodium sulphate, filtered and concentrated under reduced pressure to give the crude intermediate (R)-2-((R)-1-bromoethyl)oxirane (22 g). This was dissolved in EtOH (40 ml) before diphenylmethanamine (20 g, 109.2 mmol) and NaHCO₃(13.8 g) were added. The reaction mixture was stirred for 80 h at room temperature then heated to 80° C. for 2 h. The reaction mixture was filtered through celite bed and the filtrate concentrated under reduced pressure. The crude material was dissolved in DCM, washed with sat NH₄Cl solution, then brine, dried over sodium sulphate, filtered, concentrated and purified by silica gel column chromatography, eluting with 0-20% EtOAc in pet ether, to give the titled compound (13.0 g, 25% yield) as an oil. ¹H NMR (400 MHz, d⁶-DMSO): 7.45-7.37 (m, 4H), 7.34-7.21 (m, 2H), 7.21-7.14 (m, 2H), 5.25 (d, 1H), 4.35 (s, 1H), 3.67 (quint, 1H), 3.43 (t, 1H), 2.91 (quint, 1H), 2.43 (t, 1H), 0.62 (d, 3H).

### (2S,3R)-1-benzhydryl-3-hydroxy-2-methylazetidinium((1R,4R)-7,7-dimethyl exobicyclo[2.2.1]heptan-1-yl)methanesulfonate

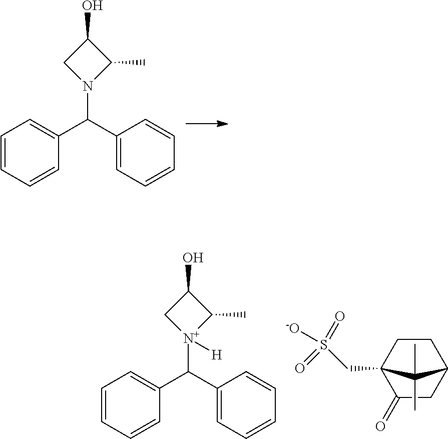

((1R,4R)-7,7-dimethyl-2-oxobicyclo[2.2.1]heptan-1-yl)methanesulfonate (11.1 g, 47.78 mol) was added to a solution of (2S,3R)-1-benzhydryl-2-methylazetidin-3-ol (11 g, 47.41 mmol) in ethanol (35 ml) at room temperature. After 12 h the reaction mixture was concentrated under reduced pressure and the crude product dissolved in DOM, EtOAc was then added, stirred for 1 h, the formed solid isolated by filtration, washed with EtOAc and dried to give the titled compound (7.5 g, 30% yield) as a solid. ¹H NMR (400 MHz, MeOD): 7.60-7.54 (m, 4H), 7.50-7.38 (m, 6H), 5.64 (s, 1H), 4.42-4.33 (m, 1H), 4.33-4.21 (m, 2H), 3.78-3.68 (m, 1H), 3.33 (d, 1H), 2.79 (d, 1H), 2.75-2.65 (m, 1H), 2.34 (dt, 1H), 2.10-1.98 (m, 2H), 1.89 (d, 1H), 1.68-1,57 (m, 1H), 1.46-1.37 (m, 1H), 1.14 (s, 3H), 1.07 (d, 3H), 0.86 (s, 3H).

### (2S,3R)-3-hydroxy-2-methylazetidinium((1R,4R)-7,7-dimethyl-2-oxobicyclo[2.2.1]heptan-1-yl)methanesulfonate

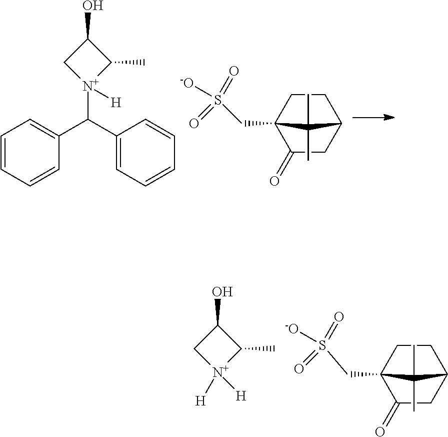

20% Pd(OH)/C (0.3 g) was added to a stirred suspension of (2S,3R)-1-benzhydryl-3-hydroxy-2-methylazetidinium ((1R,4R)-7,7-dimethyl-2-oxobicyclo[2.2.1]heptan-1-yl)methanesulfonate (5.0 g, 10.3 mmol) in methanol (25 ml) in a Hydrogenated vessel at room temperature. The reaction mixture was degassed, filled with H₂ gas (60 psi) and stirred for 16 h at room temperature. The reaction mixture was filtered through celite, washed with Methanol and the filtrate concentrated under reduced pressure. Heptane was added to the crude product, the precipitate isolated by filtration, washed with heptane and dried to give the titled compound as solid (2.4 g, 73% yield). ¹H NMR (400 MHz, MeOD): 4.35-4.20 (m, 2H), 4.04 (dd, 1H), 3.75 (dd, 1H), 2.28 (d, 1H), 2.77 (d, 1H), 2.70-2.58 (m, 1H), 2.34 (dt, 1H), 2.10-1.97 (m, 2H), 1.89 (d, 1H), 1.68-1.57 (m, 1H), 1.51 (d, 3H), 1.46-1.32 (m, 1H), 1.12 (s, 3H), 0.86 (s, 3H).

### (2S,3R)-1-(4-((1R,5S,6S)-6-((benzo[d]thiazol-2-ylthio)methyl)-3-azabicyclo[3.1.0]hexan-3-yl)-6-(trifluoromethyl)pyrimidin-2-yl)-2-methylazetidin-3-ol

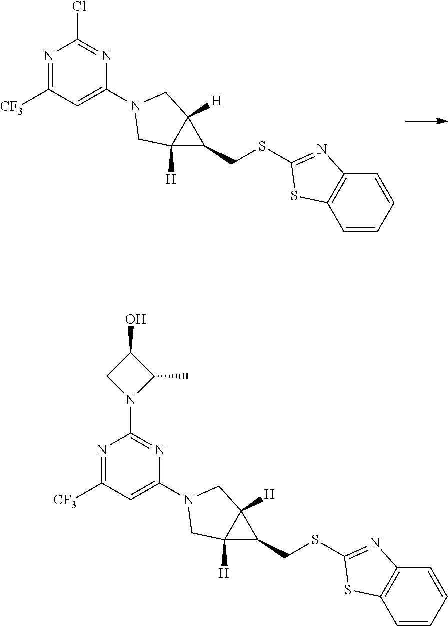 [SMILES: FC(F)(F)c1cc(N2C[C@@H]3[C@@H](CSc4nc5ccccc5s4)[C@@H]3C2)nc(Cl)n1 ; C[C@H]1[C@H](O)CN1c1nc(N2C[C@@H]3[C@@H](CSc4nc5ccccc5s4)[C@@H]3C2)cc(C(F)(F)F)n1]

DIPEA (1.8 ml) was added to a mixture of 2-(((1R,5S,6r)-3-(2-chloro-6-(trifluoromethyl)pyrimidin-4-yl)-3-azabicyclo[3.1.0]hexan-6-yl)methylthio)benzo[d]thiazole (1.5 g, 3.39 mmol) and (2S,3R)-3-hydroxy-2-methylazetidinium ((1R, 4R)-7,7-dimethyl-2-oxobicyclo[2.2.1]heptan-1-yl)methanesulfonate (1.19 g, 3.73 mmol) in MeCN (10 ml). The reaction mixture was heated at 60° C. for 20 h and then concentrated under reduced pressure. The crude material was purified by chromatography on silica gel eluting with petroleum ether/EtOAc 7.5/2.5 to give the titled compound as a solid (1.5 g, Yield: 75%). LC-MS: 3.38 mins, [M+H]⁺ 494.

### (2S,3R)-1-(4-((1R,5S,6S)-6-((benzo[d]thiazol-2-ylsulfonyl)methyl)-3-azabicyclo [3.1.0]hexan-3-yl)-6-(trifluoromethyl)pyrimidin-2-yl)-2-methylazetidin-3-ol

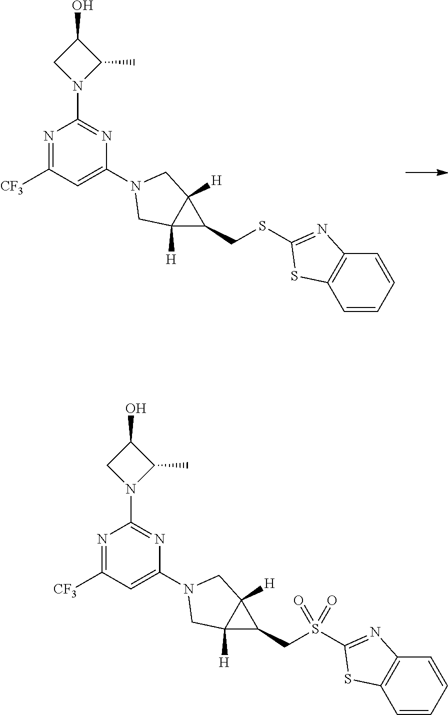 [SMILES: C[C@H]1[C@H](O)CN1c1nc(N2C[C@@H]3[C@@H](CSc4nc5ccccc5s4)[C@@H]3C2)cc(C(F)(F)F)n1 ; C[C@H]1[C@H](O)CN1c1nc(N2C[C@@H]3[C@H](C2)[C@H]3CS(=O)(=O)c2nc3ccccc3s2)cc(C(F)(F)F)n1]

Ammonium molybdate (0.75 g, 0.45 mmol) and then aq. 30% H₂O₂ (2.1 ml) dropwise, slowly, were added to a solution of (2S,3R)-1-(4-((1R,5S,6S)-6-((benzo[d]thiazol-2-ylthio)methyl)-3-azabicyclo[3.1.0]hexan-3-yl)-6-(trifluoromethyl)pyrimidin-2-yl)-2-methylazetidin-3-ol (1.5 g, 3.04 mmol) in ethanol (30 ml) at 0° C. The reaction mixture was allowed to warm to room temperature and stirred for a further 5 h. Water was added, the precipitated solid isolated by filtration and purified by silica gel column chromatography eluting with 30% pet ether in ethyl acetate to give the titled compound as a solid (0.8 g, Yield: 50%). ¹H NMR (400 MHz, CLCl₃): 8.22 (d, 1H), 8.05 (d, 1H), 7.69-7.58 (m, 2H), 5.90 (s, 1H), 4.31-4.08 (m, 3H), 4.07-3.75 (bm, 1H), 3.78-3.68 (m, 1H), 3.60-3; LC-MS: 4.13 mins, [M+H]⁺ 526.

### Sodium((1R,5S,6S)-3-(2-((2S,3R)-3-hydroxy-2-methylazetidin-1-yl)-6-(trifluoromethyl)pyrimidin-4-yl)-3-azabicyclo[3.1.0]hexan-6-yl)methanesulfinate

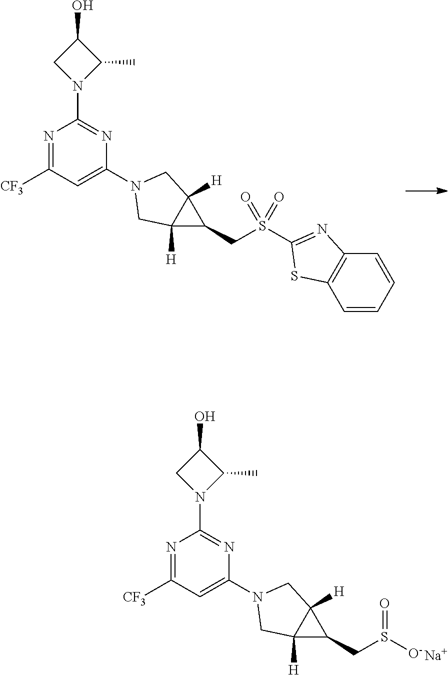 [SMILES: C[C@H]1[C@H](O)CN1c1nc(N2C[C@@H]3[C@H](C2)[C@H]3CS(=O)(=O)c2nc3ccccc3s2)cc(C(F)(F)F)n1 ; C[C@H]1[C@H](O)CN1c1nc(N2C[C@@H]3[C@H](C2)[C@H]3CS(=O)[O-])cc(C(F)(F)F)n1 ; [Na+]]

0.5M sodium methoxide solution in methanol (1.14 ml, 0.57 mmol) was slowly added dropwise to a solution of (2S,3R)-1-(4-((1R,5S,6S)-6-((benzo[d]thiazol-2-ylsulfonyl)methyl)-3-azabicyclo[3.1.0]hexan-3-yl)-6-(trifluoromethyl)pyrimidin-2-yl)-2-methylazetidin-3-ol (0.3 g, 0.57 mmol) in methanol (3 ml) at 0° C. The reaction mixture was allowed to warm to room temperature, stirred for a further 3 h, concentrated under reduced pressure and a slurry made with celite. The slurry was filtered, washed with 0-30% methanol in diethyl ether, the filtrates combined and concentrated to afford the titled compound (0.15 g, Yield: 63.5%); ¹H NMR (400 MHz, d⁶-DMSO): 4.09-3.95 (m, 2H), 3.95-3.70 (m, 2H), 3.60-3.50 (m, 1H), 3.49-3.33 (m, 3H), 1.88-1.78 (m, 2H), 1.54 (bs, 2H), 1.45-1.32 (m, 3H), 0.65-0.58 (m, 1H); LC-MS: 3.35 mins, [M+H]E 393.

### Example 4—Preparation of sodium((1R,5S,6S)-3-(2-((S)-2-methylpyrrolidin-1-yl)-6-(trifluoromethyl)pyrimidin-4-yl)-3-azabicyclo[3.1.0]nexan-6-yl)methanesulfinate

### 2-((((1R,5S,6S)-3-(2-((S)-2-methylpyrrolidin-1-yl)-6-(trifluoromethyl)pyrimidin-4-yl)-3-azabicyclo[3.1.0]hexan-6-yl)methypthio)benzoidithiazole

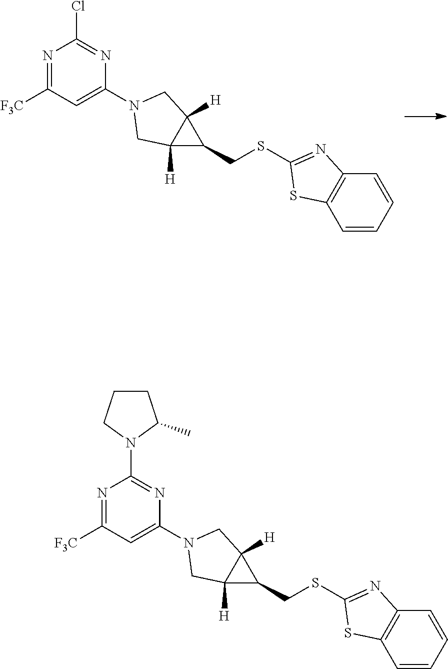 [SMILES: FC(F)(F)c1cc(N2C[C@@H]3[C@@H](CSc4nc5ccccc5s4)[C@@H]3C2)nc(Cl)n1 ; C[C@H]1CCCN1c1nc(N2C[C@@H]3[C@@H](CSc4nc5ccccc5s4)[C@@H]3C2)cc(C(F)(F)F)n1]

(S)-2-Methylpyrrolidine (2.88 g, 33.82 mmol) and DIPEA (11.8 mL, 67.73 mmol) were added to a stirred suspension of 2-(((1R,5S,6r)-3-(2-chloro-6-(trifluoromethyl)pyrimidin-4-yl)-3-azabicyclo[3.1.0]hexan-6-yl)methylthio)benzo[d]thiazole (10.0 g, 22.57 mmol) in acetonitrile (100 mL). After 16 h at 60° C. the reaction was cooled to room temperature, quenched with water, extracted with ethyl acetate. The combined organic layers were washed with water, brine, dried over anhydrous sodium sulphate, filtered and concentrated to dryness. The crude product was washed with n-pentane, filtered and dried to the titled compound (10 g, 90%) as a solid. LC-MS: 2.97 mins, [M+H]⁺ 492

### 2-((((1R,5S,6S)-3-(2-((S)-2-methylpyrrolidin-1-yl)-6-(trifluoromethyl)pyrimidin-4-yl)-3-azabicyclo[3.1.0]hexan-6-yl)methyl)sulfonyl)benzo[d]thiazole

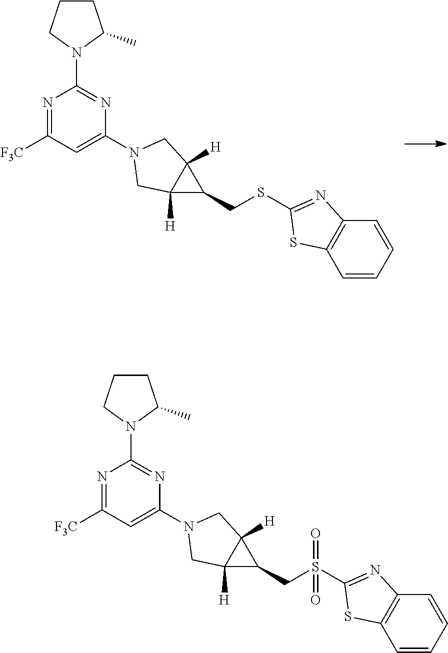 [SMILES: C[C@H]1CCCN1c1nc(N2C[C@@H]3[C@@H](CSc4nc5ccccc5s4)[C@@H]3C2)cc(C(F)(F)F)n1 ; C[C@H]1CCCN1c1nc(N2C[C@@H]3[C@H](C2)[C@H]3CS(=O)(=O)c2nc3ccccc3s2)cc(C(F)(F)F)n1]

Ammonium molybdate (6.0 g, 30.51 mmol) and hydrogen peroxide (30% in water) solution (16 ml, 141.1 mmol) were added to a solution of 2-((((1R,5S,6S)-3-(2-((S)-2-methylpyrrolidin-1-yl)-6-(trifluoromethyl)pyrimidin-4-yl)-3-azabicyclo[3.1.0]hexan-6-yl)methyl)thio)benzo[d]thiazole (10.0 g, 20.3 mmol) in ethanol (250 ml) at 0° C. and the reaction mixture was then allowed to warm to room temperature. After 16 h the reaction was filtered, washed with ethanol, dried under vacuum and purified by flash column chromatography on silica gel eluting with 7% ethyl acetate in petroleum ether to give the titled compound (5.3 g, 49%) as a solid, LC-MS: 2.57 mins, [M+H]⁺ 524.

### Sodium((1R,5S,6S)-3-(2-((S)-2-methylpyrrolidin-1-yl)-6-(trifluoromethyl)pyrimidin-4-yl)-3-azabicyclo[3.1.0]hexan-6-yl)methanesulfinate

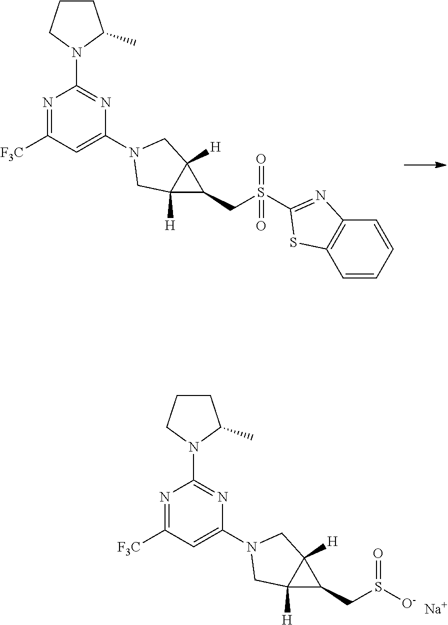 [SMILES: C[C@H]1CCCN1c1nc(N2C[C@@H]3[C@H](C2)[C@H]3CS(=O)(=O)c2nc3ccccc3s2)cc(C(F)(F)F)n1 ; C[C@H]1CCCN1c1nc(N2C[C@@H]3[C@H](C2)[C@H]3CS(=O)[O-])cc(C(F)(F)F)n1 ; [Na+]]

A solution of 0.5 M sodium methoxide in methanol (16 ml, 8.021 mmol) was added drop wise to a mixture of 2-((((1R,5S,6S)-3-(2-(((S)-2-methylpyrrolidin-1-yl)-6-(trifluoromethyl)pyrimidin-4-yl)-3-azabicyclo[3.1.0]hexan-6-yl)methyl)sulfonyl)benzo[d]thiazole (4.2 g, 8.02 mmol) in methanol (35 ml) and tetrahydrofuran (18 ml). The reaction was then warmed to room temperature and after 16 h concentrated under vacuum. The crude product was washed with n-pentane, filtered and dried to give the titled compound (2.11 g, 64%) as a solid. %). ¹H NMR (400 MHz, d⁶-DMSO): 6.05 (5, 1H), 4.18-4.10 (m, 1H), 3.95-3.75 (m, 1H), 3.59-3.30 (m, 5H), 2.10-1.70 (m, 5H), 1.67-1.49 (m, 3H), 1.3-1.1 (m, 3H), 0.65-0.55 (m, 1H); LC-MS: 1.97 mins, [M+H]^{+ 391.}

### Example 5—Preparation of Sodium((1R,5S,6S)-3-(2-((S)-2-methylpiperidin-1-yl)-6-(trifluoromethyl)pyrimidin-4-yl)-3-azabicyclo[3.1.0]hexan-6-yl)methanesulfinate

### 2-((((1R,5S,6S)-3-(2-((S)-2-methylpiperidin-1-yl)-6-(trifluoromethyl)pyrimidin-4-y)-3-azabicyclo[3.1.0]hexan-6-yl)methyl)thio)benzo[d]thiazole

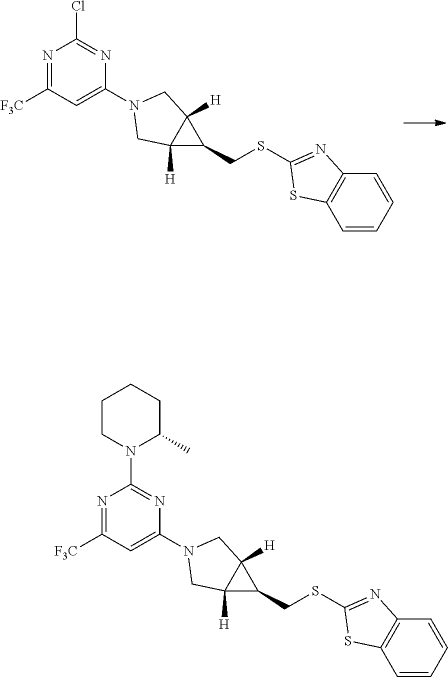 [SMILES: FC(F)(F)c1cc(N2C[C@@H]3[C@@H](CSc4nc5ccccc5s4)[C@@H]3C2)nc(Cl)n1 ; C[C@H]1CCCCN1c1nc(N2C[C@@H]3[C@@H](CSc4nc5ccccc5s4)[C@@H]3C2)cc(C(F)(F)F)n1]

(S)-2-Methylpiperidine (2.04 ml, 16.92 mmol) and DIPEA (7.86 mL, 45.15 mmol) were added to a stirred suspension of 2-(((1R,5S,6r)-3-(2-chloro-6-(trifluoromethyl)pyrimidin-4-yl)-3-azabicyclo[3.1.0]hexan-6-yl)methylthio)benzo[d]thiazole (5.0 g, 11.28 mmol) in acetonitrile (50 ml) and the reaction mixture heated to 80° C. More (S)-2-methylpiperidine (5.44 ml, 45.15 mmol) was then added in four equal portions every 14 h. The reaction mixture was cooled to room temperature, quenched with water and extracted with ethyl acetate. The combined organic layers were washed with water, brine, dried over anhydrous sodium sulphate, filtered, and concentrated to dryness. The crude product was washed with n-pentane, filtered and dried to give the titled compound (5.8 g) as a solid. LC-MS: 3.06 mins, [M+H]⁺506.

### 2-((((1R,5S,6S)-3-(2-((S)-2-methylpiperidin-1-yl)-6-(trifluoromethyl)pyrimidin-4-yl)-3-azabicyclo[3.1.0]hexan-6-yl)methyl)sulfonyl)benzofdithiazole

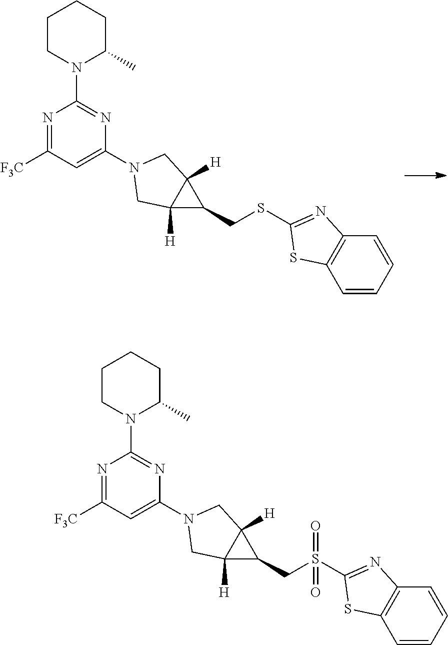 [SMILES: C[C@H]1CCCCN1c1nc(N2C[C@@H]3[C@@H](CSc4nc5ccccc5s4)[C@@H]3C2)cc(C(F)(F)F)n1 ; C[C@H]1CCCCN1c1nc(N2C[C@@H]3[C@H](C2)[C@H]3CS(=O)(=O)c2nc3ccccc3s2)cc(C(F)(F)F)n1]

Ammonium molybdate (3.37 g, 17.20 mmol) and hydrogen peroxide (30% in water) solution (9 ml, 79.71 mmol) were added to a solution of 2-((((1R,5S,6S)-3-(2-((S)-2-methylpiperidin-1-yl)-6-(trifluoromethyl)pyrimidin-4-yl)-3-azabicyclo[3.1.0]hexan-6-yl)methyl)thio)benzo[d]thiazole (5.8 g, 11.47 mmol) in ethanol (180 ml) at 0° C. The reaction mixture was then allowed to warm at room temperature. After 16 h the reaction mixture was filtered, washed with ethanol, dried under vacuum and purified by flash column chromatography on silica gel eluting with 7% ethyl acetate in petroleum ether to give the titled compound (4.0 g, 65%) as a solid. LC-MS: 2.71 mins, [M+HJ]⁺538.

### Sodium((1R,5S,6S)-3-(2-((S)-2-methylpiperidin-1-yl)-6-(trifluoromethyl)pyrimidin-4-yl)azabicyclo[3.1.0]hexan-6-yl)methanesulfinate

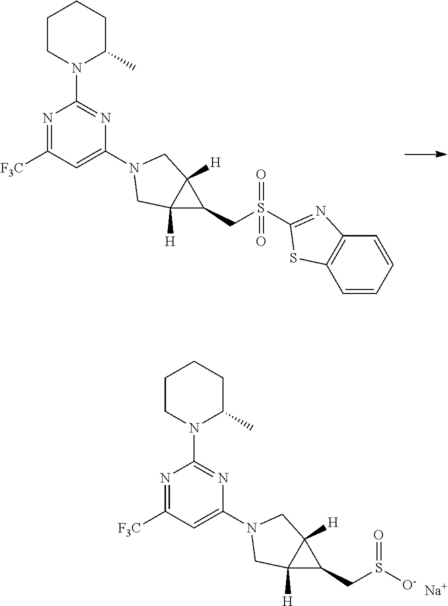 [SMILES: C[C@H]1CCCCN1c1nc(N2C[C@@H]3[C@H](C2)[C@H]3CS(=O)(=O)c2nc3ccccc3s2)cc(C(F)(F)F)n1 ; C[C@H]1CCCCN1c1nc(N2C[C@@H]3[C@H](C2)[C@H]3CS(=O)[O-])cc(C(F)(F)F)n1 ; [Na+]]

A solution of 0.5 M sodium methoxide in methanol (11.16 ml, 5.58 mmol) was added dropwise to a stirred solution of 2-((((1R,5S,6S)-3-(2-((S)-2-methylpiperidin-1-yl)-6-(trifluoromethyl)pyrimidin-4-yl)-3-azabicyclo[3.1.0]hexan-6-yl)methyl)sulfonyl)benzo[d]thiazole

(3 g, 5.58 mmol) in methanol (24 ml) and tetrahydrofuran (12 ml) at 0° C. The reaction mixture was warmed to room temperature and after 8 h the reaction mixture was concentrated under vacuum, washed with n-pentane and dried to give the titled compound (2.1 g, 88%) as a solid. ¹H NMR (400 MHz, d⁶-DMSO); 6.04 (s, 1H), 4.98-4.87 (m, 1H), 4.55-4.45 (m, 1H), 3.88-3.72 (m, 1H), 3.62-3.48 (m, 1H), 3.47-3.35 (m, 1H), 2.90-2.78 (m, 2H), 1.85-1.72 (m, 2H), 1.70-1.48 (m, 6H), 1.40-1.20 (m, 1H), 1.10 (d, 3H), 0.66-0.55 (m, 1H); LC-MS: 2.18 mins, [M+H]⁺405.

### Example 6—Preparation of sodium((1R,5S,6S)-3-(5-methyl-2-((S)-2-methylazetidin-1-yl)-6-(trifluoromethyl)pyrimidin-4-yl)-3-azabicyclo[3.1.0]hexan-6-yl)methanesulfinate

### 5-Methyl-6-(trifluoromethyl)pyrimidine-2,4-diol

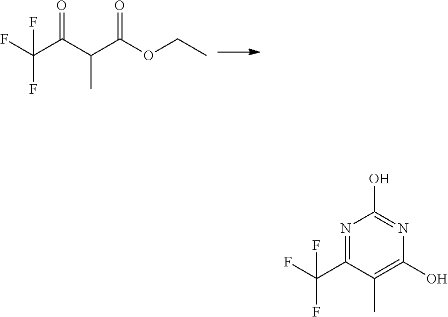

A solution of 21% sodium ethoxide in ethanol (16.3 mL, 50.4 mmol) was added dropwise to a stirred solution of ethyl 2-methyl-4,4,4-trifluoroacetoacetate (5 g, 25.2 mmol) and urea (1.81 g, 30.3 mmol) in toluene (35 ml) at 0° C. After 30 mins the mixture was heated at 120° C. for 48 h and then concentrated to dryness to afford the titled compound (5 g) as a solid which was used in the next step without purification. LC-MS: 1.05 mins, [M+H]⁺ 195

### 2-(((1R,5S,6r)-3-(5-methyl-2-chloro-6-(trifluoromethyl)pyrimidin-4-yl)-3-azabicyclo[3.1.0]hexan-6-yl)methylthio)benzo[d]thiazole

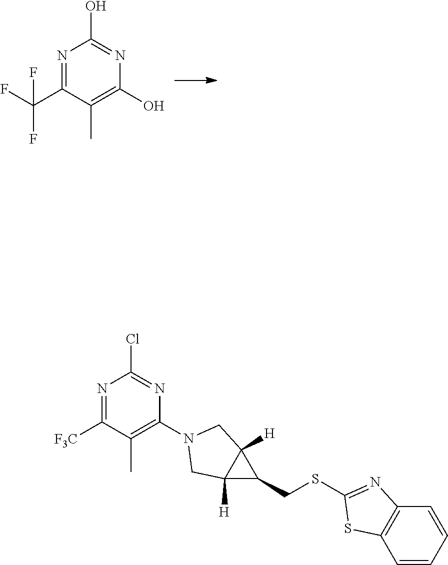 [SMILES: Cc1c(O)nc(O)nc1C(F)(F)F ; Cc1c(N2C[C@@H]3[C@@H](CSc4nc5ccccc5s4)[C@@H]3C2)nc(Cl)nc1C(F)(F)F]

5-Methyl-6-(trifluoromethyl)pyrimidine-2,4-diol (10 g, 51.52 mmol) was added to a mixture of dimethyl aniline (6.5 ml, 51.51 mmol) in phosphorus oxychloride (34 ml) at 0° C. and then heated at 100° C. After 16 h the mixture was poured over ice water and extracted with MTBE. The combined organic layers were washed with water, brine, dried over anhydrous sodium sulphate, filtered and concentrated to dryness. The crude product was washed with n-hexane, decanted and the decanted n-hexane evaporated to dryness to give 2,4-dichloro-5-methyl-6-(trifluoromethyl)pyrimidine (3.8 g, 32%) as a liquid, which was used in the next step without further purification.

The 2,4-dichloro-5-methyl-6-(trifluoromethyl)pyrimidine (3.8 g, 16.45 mmol) was dissolved in dichloromethane (10 ml) and added drop wise to a suspension of 2-((1 R,5S,6r)-3-azabicyclo[3.1.0]hexan-6-ylmethylthio)benzo[d]thiazole hydrochloride (4.42 g, 14.8 mmol) in dichloromethane (30 ml) of at 0° C. Following the addition of DIPEA (10 ml, 57.5 mmol) the reaction mixture was then warmed to room temperature. After 5 h the reaction was quenched with water, extracted with dichloromethane, the combined organic layers washed with water, brine, dried over anhydrous sodium sulphate, filtered and concentrated to dryness. The crude product was the washed with n-pentane, diethyl ether, filtered and dried to afford the titled compound (5.1 g, 68%) as a solid. ¹H NMR (400 MHz, CDCl₃): 7.86 (d, 1H), 7.76 (d, 1H), 7.42 (t, 1H), 7.30 (t, 1H), 4.13 (d, 2H), 3.73 (d, 2H), 3.39 (d, 2H), 2.31 (5, 3H), 1.78 (s, 2H), 1.21-1.12 (m, 1H); LC-MS: 2.60 mins, [M+H]⁺457

### 2-((((1R,5S,6S)-3-(5-methyl-2-((S)-2-methylazetidin-1-yl)-6-(trifluoromethyl)pyrimidin 4-3-azabicyclo [3.1.0]hexan-6-yl)methyl)thio)benzo[d]thiazole

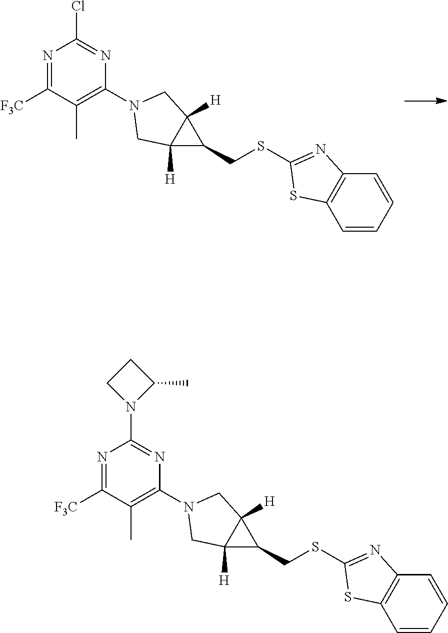 [SMILES: Cc1c(N2C[C@@H]3[C@@H](CSc4nc5ccccc5s4)[C@@H]3C2)nc(Cl)nc1C(F)(F)F ; Cc1c(N2C[C@@H]3[C@@H](CSc4nc5ccccc5s4)[C@@H]3C2)nc(N2CC[C@@H]2C)nc1C(F)(F)F]

DIPEA (2.2 ml) was added to a solution of 2-(((1 R,5S,6r)-3-(2-chloro-6-(trifluoromethyl)pyrimidin-4-yl)-3-azabicyclo[3.1.0]hexan-6-yl)methylthio)benzo[d]thiazole (2.0 g, 4.38 mmol) and (S)-2-methylazetidinium ((1R,4R)-7,7-dimethyl-2-oxobicyclo[2.2.1]heptan-1-yl)methanesulfonate (1.8 g, 5.9 mmol) in acetonitrile (20 mL) to and heated at 60° C. for 12 h. The reaction mixture was cooled to room temperature, quenched with water and extracted with ethyl acetate. The combined organic layer were washed with water, brine, dried over anhydrous sodium sulphate, filtered and concentrated to dryness. The crude product was washed with n-pentane, filtered and dried to give the titled compound (1.9 g, 88%) as a solid. LC-MS: 2.93 mins, [M+H]⁺492

### 2-((((1R,5S,6S)-3-(5-methyl-2-((S)-2-methylazetidin-1-yl)-6-(trifluoromethyl)pyrimidin-4-yl)-3-azabicyclo[3.1.0]hexan-6-yl)methyl)sulfonyl)benzordithiazole

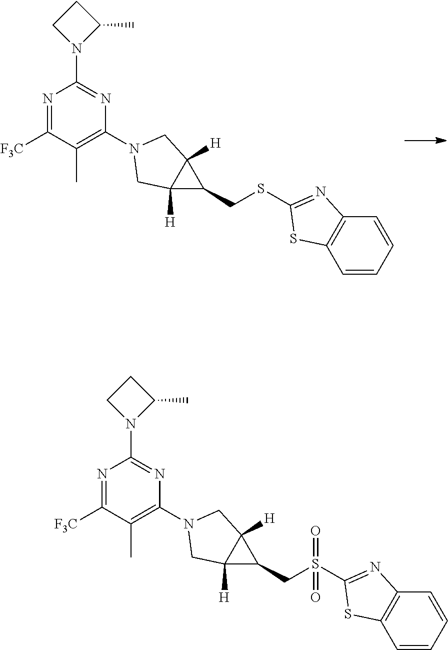 [SMILES: Cc1c(N2C[C@@H]3[C@@H](CSc4nc5ccccc5s4)[C@@H]3C2)nc(N2CC[C@@H]2C)nc1C(F)(F)F ; Cc1c(N2C[C@@H]3[C@H](C2)[C@H]3CS(=O)(=O)c2nc3ccccc3s2)nc(N2CC[C@@H]2C)nc1C(F)(F)F]

Ammonium molybdate (3.82 g, 19.5 mmol) and 30% Aq. H₂O₂(11 mL, 98.97 mmol) were added to a solution of 2-((((1R,5S,6S)-3-(5-methyl-2-((S)-2-methylazetidin-1-yl)-6-(trifluoromethyl)pyrimidin-4-yl)-3-azabicyclo[3.1.0]hexan-6-yl)methyl)thio)benzo[d]thiazole (7 g, 14.24 mmol) in ethanol (150 ml) at 0° C. were added. The reaction mixture was warmed to room temperature and after 16 h filtered, washed with ethanol, dried under vacuum and purified by flash column chromatography on silica gel eluting with 7% ethyl acetate in pet ether to give the titled compound as a solid (4 g, 53%). LC-MS: 2.52 mins, [M+H]⁺524

### Sodium((1R,5S,6S)-3-(5-methyl-2-((S)-2-methylazetidin-1-yl)-6-(trifluoromethyl)pyrimidin-4-yl)-3-azabicyclo[3.1.0]hexan-6-yl)methanesulfinate

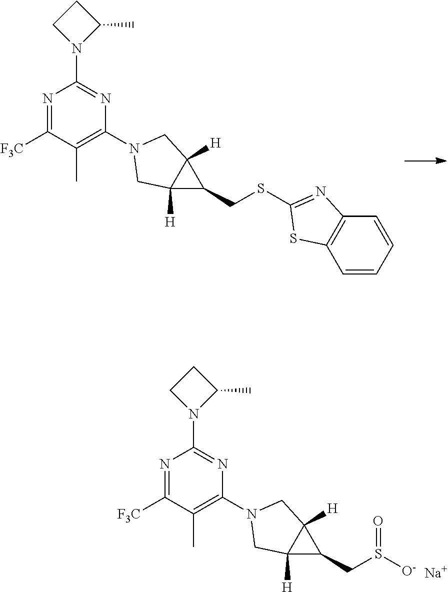 [SMILES: Cc1c(N2C[C@@H]3[C@@H](CSc4nc5ccccc5s4)[C@@H]3C2)nc(N2CC[C@@H]2C)nc1C(F)(F)F ; Cc1c(N2C[C@@H]3[C@H](C2)[C@H]3CS(=O)[O-])nc(N2CC[C@@H]2C)nc1C(F)(F)F ; [Na+]]

A solution of 0.5 M sodium methoxide solution in methanol (8.4 ml, 4.2 mmol) was added to a stirred solution of 2-((((1R,5S,6S)-3-(5-methyl-2-((S)-2-methylazetidin-1-yl)-6-(trifluoromethyl)pyrimidin-4-yl)-3-azabicyclo[3.1.0]hexan-6-yl)methyl)sulfonyl)benzo[d]thiazole (2.2 g, 4.2 mmol) in methanol (10 ml) and tetrahydrofuran (3 ml) at 0° C. The reaction mixture was allowed to warm at room temperature and after 16 h concentrated under vacuum. The crude product was washed with n-pentane, filtered and dried to give the titled compound (1.8 g, 85%) as solid. ¹H NMR (400 MHz, d⁶-DMSO): 4.25-4.35 (m, 1H), 4.02-3.73 (m, 4H), 3.66-3.48 (m, 1H), 2.38-2.26 (m, 1H), 2.14 (s, 3H), 1.94-1.82 (1H), 1.95-1.82 (m, 2H), 1.76 (d, 2H), 1.47 (s, 2H), 1.41 (d, 3H), 0.68-0.59 (m, 1H); LC-MS: 3.81 mins, [M+H]⁺391

### Example 7—Assessing Metabolite Formation

The compound is incubated with liver hepatocytes (for example primary human hepatocytes) in an incubator at 37° C. The reaction is stopped at 15, 30, 60, 90, 120 min by precipitating a sample of incubation mixture with acetonitrile. The precipitated sample is subjected to subsequent analysis by HPLC and mass spectrometry to ascertain which metabolites are formed. The analysis is expected to show oxidation of the parent compound to give hydroxy derivatives, but no presence of acyl-glucuronide like metabolites in which a glucuronide group has been added to the sulfinic acid functionality.

### Example 8—Determining the Dose Response and IC₅₀ of Compound Examples 2 and 6 with Human Ketohexokinase (KHK-A)

The following materials and methods were used,
Enzyme description and source: Recombinant Human Ketohexokinase, Met1Val298, with C terminal 6His tag (R&D Systems, 8177-HK).
Reagents: 200 μM ATP and 100 mM D-(−)-Fructose (Sigma F0127) as substrates.
Enzyme reaction buffer: 25 mM HEPES, 300 mM KCl, 10 mM MgCl₂, 10 mM CaCl2, pH 7.0.
Control compound: KHK Inhibitor (Millipore-Sigma 420640) See Maryanoff, B. E., et al. 2011. ACS Med. Chem. Lett. 2, 538.
Assay: Transcreener Kinase ADP2 FP assay as described: https://www.bellbrooklabs.com/products/transcreener-hts-assays/kinase-assays/
Assay parameters: 12-point dose response (n=2), 3-fold serial dilution, starting at 100 μM

KHK-A was added to the tested compounds (from 10 mM stock solution in 25 mM Tris pH 7.4), to a total volume 5 μl, and pre-incubated for 30 minutes at rt to ensure E*I complex formation. The reaction was then initiated by addition of an equal volume (5 μl) containing ATP and Fructose to give a resulting mix of 17.5 nM KHK-A, 0.3 mM ATP, 7 mM Fructose, 25 mM Tris (pH 7.4), 10 mM MgCl₂, 10 mM CaCl₂ and 50 mM KCl. Following 60 min at 30° C., reactions were quenched with 10 μl of stop/detect mix (40 mM HEPES (pH 7.5), 80 mM EDTA, 0,04% Brij-35, 8 nM ADP tracer and optimal concentration of ADP2 antibody), The plates were read on a PHERAstar PLUS after 1-hour room temperature equilibration. Non-linear regression, variable slope inhibitor curve fits (Prism, Graph-Pad) were used to determine the IC₅₀ of the compounds where possible. Results are shown in table 1.

TABLE 1
inhibition of human KHK-A by compound examples 2 and 6

| Example | IC₅₀/nM |
|---|---|
| 2 | 740 |
| 6 | 78 |

### Example 9—Effect of Examples 2 and 6 on Oral Fructose Tolerance Test (OFTT) in male Sprague Dawley Rats

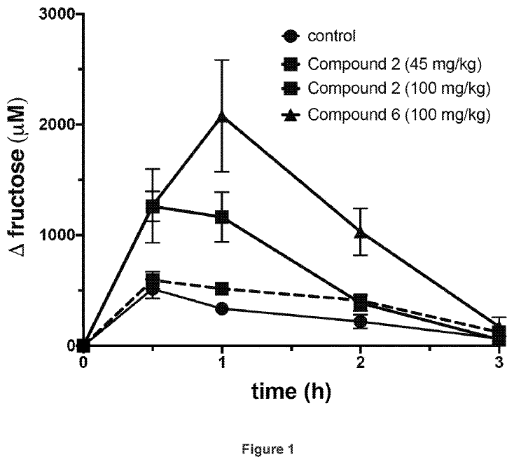

The animals were fasted overnight, basal blood samples collected and a 100 mg/kg dose was orally administered of compounds example 2 or example 6. A fructose bolus (2 g/kg) was then given 1 hour after test compound administration and blood samples collected at 0.5, 1, 2 and 3 hours post fructose challenge. All the blood samples were centrifuged immediately and the separated plasma samples were stored at appropriate temperature for the fructose estimation. Plasma fructose concentration was measured by commercially available EnzyChrome fructose assay kit. Results are shown in FIG. 1.

1. A compound having a structure represented by Formula A: 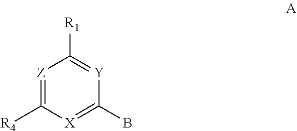
wherein
Z is CH, N or C—CN;
Y is N or CH;
X is N or CR₃;
B is 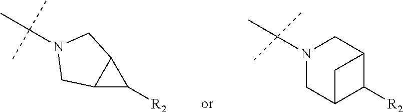
R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein said —C₁₋₃ alkyl is substituted with 0 to 3 halogen atoms, and provided that there is no more than one —OH substituent; or
R₁ is —N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;
R₂ is -(L)ₘ-S(O)OH, -L-(CH₂)ₙS(O)OH, -(L)ₘ-S(O)₂OH, -L-(CH₂)ₙS(O)₂OH;
m is 0 or 1;
n is 0 or 1;
L is CH₂, CHF, or CF₂;
R₃ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;
R₄ is —C₁₋₃ alkyl or —C₃₋₄ cycloalkyl, wherein the C₁₋₃ alkyl or —C₃₋₄ cycloalkyl group is optionally substituted with 0 to 5 halogen atoms;
or R₃ and R₄ come together with the carbon atoms to which they are attached to form a C₄₋₇ cycloalkyl ring wherein each carbon atom in the ring is substituted by 0 to 2 R₅ groups;
R₅ is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;
or an enantiomer, stereoisomer, tautomer, solvate, hydrate, amino acid conjugate, or pharmaceutically acceptable salt thereof

2. The compound of claim 1, wherein the compound has a structure represented by Formula B: 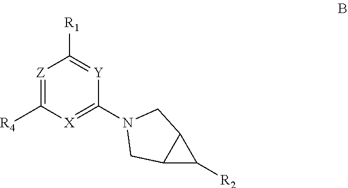
wherein
Z is CH, N or C—CN;
Y is N or CH;
X is N or CR₃;
R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R₂ is -(L)ₘ-S(O)OH, -L-(CH₂)ₙS(O)OH, -(L)ₘ-S(O)₂OH, -L-(CH₂)ₙS(O)₂OH;
m is 0 or 1;
n is 0 or 1;
L is CH₂, CHF, or CF₂;
R₃ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R₄ is cyclopropyl, cyclobutyl, or —C₁₋₃ alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof

3. The compound of claim 1, wherein the compound has a structure represented by Formula B1: 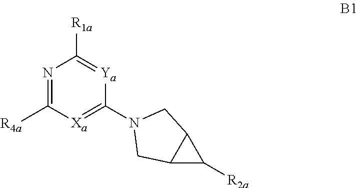
wherein
Yₐ is N or CH;
Xₐ is N or CR₃ₐ;
R₁ₐ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R₂ₐ is -(L)ₘ-S(O)OH, -L-(CH₂)ₙS(O)₂OH, -(L)ₘ-S(O)₂OH, -L-(CH₂)ₙS(O)₂OH;
m is 0 or 1;
n is 0 or 1;
L is CH₂, CHF, or CF₂;
R₃ₐ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R₄ₐ is cyclopropyl, cyclobutyl, or —C₁₋₃ alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof

4. The compound of claim 1, wherein the compound has a structure represented by Formula B2: 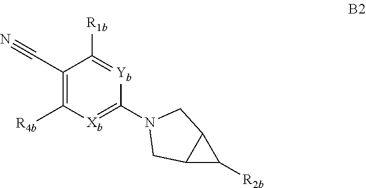
wherein
Y_{b} is N or CH;
Xb is N or CR_{3b};
R_{1b} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R_{2b} is -(L)ₘ-S(O)OH, -L-(CH₂)ₙS(O)OH, -(L)ₘ-S(O)₂OH or -L-(CH₂)ₙS(O)₂OH;
m is 0 or 1;
L is CH₂, CHF, or CF₂;
R_{3b} is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R_{4b} is cyclopropyl, cyclobutyl, or —C₁₋₃alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof

5. The compound of claim 1, wherein the compound has a structure represented by Formula B3: 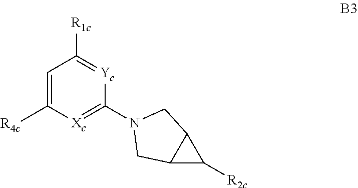
wherein
Y_{c} is N or CH;
X_{c} is N or CR_{3c};
R_{1c} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH sub stituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R_{2c} is -(L)ₘ-S(O)OH, -L-(CH₂)ₙS(O)OH, -(L)ₘ-S(O)₂OH or -L-(CH₂)ₙS(O)₂OH;
m is 0 or 1;
n is 0 or 1;
L is CH₂, CHF, or CF₂;
R_{3c} is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R_{4c} is cyclopropyl, cyclobutyl, or —C₁₋₃alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof

6. The compound of claim 1, wherein the compound has a structure represented by Formula C: 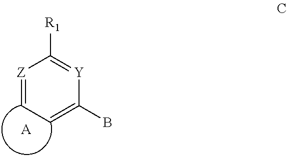
wherein
Z is CH, N or C—CN;
Y is N or CH;
Ring A is a C₄₋₇ cycloalkyl ring wherein each carbon atom in the ring is substituted by 0 to 2 R₅ groups;
B is 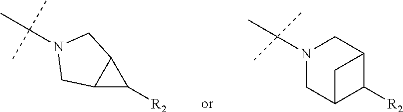
R₁ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein said —C₁₋₃ alkyl is substituted with 0 to 3 halogen atoms, and provided that there is no more than one —OH substituent; or
R₁ is —N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;
R₂ is -(L)ₘ-S(O)OH, -L-(CH₂)ₙS(O)OH, -(L)ₘ-S(O)₂OH, -L-(CH₂)ₙS(O)₂OH;
m is 0 or 1;
n is 0 or 1;
L is CH₂, CHF, or CF₂;
R₅ is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;
or an enantiomer, stereoisomer, tautomer, solvate, hydrate, amino acid conjugate, or pharmaceutically acceptable salt thereof.

7. The compound of claim 1, wherein the compound has a structure represented by Formula C1: 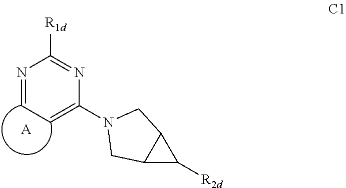
wherein
Ring A is a C₄₋₇ cycloalkyl ring wherein each carbon atom in the ring is substituted by 0 to 2 R₅ groups
R_{1d} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein —C₁₋₃ alkyl is substituted with 0 to 3 halogen atoms, and provided that there is no more than one —OH substituent; or
R_{1d} is —N(C₁₋₃alkyl)₂, —NH(C₁₋₃ alkyl), or —NH(C₃₋₄ cycloalkyl), wherein each C₁₋₃ alkyl or C₃-cycloalkyl is substituted with 0 to 1 —OH substituent;
R_{2d} is -(L)ₘ-S(O)OH, -L-(CH₂)ₙS(O)OH, -(L)ₘ-S(O)₂OH, -L-(CH₂)ₙS(O)₂OH;
m is 0 or 1;
n is 0 or 1;
L is CH₂, CHF, or CF₂;
R_{5d} is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;
or an enantiomer, stereoisomer, tautomer, solvate, hydrate, amino acid conjugate, or pharmaceutically acceptable salt thereof

8. The compound of claim 3, wherein
Yₐ is N;
Xₐ is CR₃ₐ;
R₁ₐ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent;
R₂ₐ is —CH₂—S(O)OH or —(CH₂)₂—S(O)OH;
R₁ₐ is H, halogen or —C₁₋₃ alkyl optionally substituted with 1 to 3 halogen atoms; and
R₄ₐ is —C₁₋₃ alkyl optionally substituted with 0 to 5 halogen atoms.

9. The compound of claim 1, which is sodium((1R,5S,6S)-3-(2-((S)-2-methylazetidin-1-yl)-6-(trifluoromethyl)pyrimidin-4-yl)-3-azabicyclo[3.1.0]hexan-6-yl)methanesulfinate, sodium((1R,5S,6S)-3-(2-((2S,3R)-3-hydroxy-2-methylazetidin-1-yl)-6-(trifluoromethyl)pyrimidin-4-yl)-3-azabicyclo[3.1.0]hexan-6-yl)methanesulfinate, sodium ((1R,5S,6S)-3-(2-((S)-2-methylpyrrolidin-1-yl)-6-(trifluoromethyl)pyrimidin-4-yl)-3-azabicyclo[3.1.0]hexan-6-yl)methanesulfinate, sodium((1R,5S,6S)-3-(2-((S)-2-methylpiperidin yl)-6-(trifluoromethyl)pyrimidin-4-yl)-3-azabicyclo[3.1.0]hexan-6-yl)methanesulfinate, sodium ((1R,5S,6S)-3-(5-methyl-2-((S)-2-methylazetidin-1-yl)-6-(trifluoromethyl)pyrimidin-4-yl)azabicyclo[3.1.0]hexan-6-yl)methanesulfinate or sodium((1R,5S,6S)-3-(2-((S)-2-methylazetidin-1-yl)-8,8-difluoro-5,6,7,8-tetrahydroquinazolin-4-yl)-3-azabicyclo[3.1.0]hexan-6-yl)methanesulfinate.

10. A pharmaceutical composition comprising:
a compound of claim 1; and
at least one of a pharmaceutically acceptable excipient, diluent or carrier.

11. (canceled)

12. A method of inhibiting ketohexokinase (KHK), the method comprising administering to a subject in need thereof a compound of claim 1.

13. A medicament for inhibiting ketohexokinase (KHK) comprising the compound of claim 1.

14. A method of treating and/or preventing a disease or disorder in which ketohexokinase (KHK) plays a role, the method comprising administering to a subject in need thereof an effective amount of claim 1.

15. A medicament for treating and/or preventing a disease or disorder in which ketohexokinase (KHK) plays a role, comprising the compound of claim 1.

16. The method of claim 14, wherein the disease or disorder is selected from the group consisting of T1D, T2D, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, acute kidney disorder, tubular dysfunction, proinflammatory changes to the proximal tubules, diabetic retinopathy, adipocyte dysfunction, visceral adipose deposition, obesity, eating disorders, excessive sugar craving, dyslipidemia, hyperlipidemia, hypertriglyceridemia, increased total cholesterol, high LDL cholesterol, low HDL cholesterol, hyperinsulinemia, NAFLD, steatosis, NASH, fibrosis, cirrhosis, hepatocellular carcinoma, HFI, coronary artery disease, peripheral vascular disease, hypertension, endothelial dysfunction, impaired vascular compliance, congestive heart failure, myocardial infarction, stroke, hemorrhagic stroke, ischemic stroke, pulmonary hypertension, restenosis after angioplasty, intermittent claudication, post-prandial lipemia, metabolic acidosis, ketosis, arthritis, osteoporosis, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, foot ulcerations, ulcerative colitis, hyper apo B lipoproteinemia, Alzheimer's Disease, schizophrenia, impaired cognition, inflammatory bowel disease, ulcerative colitis, Crohn's disease, and irritable bowel syndrome.

17. The compound of claim 2, wherein the compound has a structure represented by Formula B1: 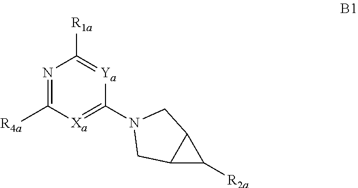
wherein
Yₐ is N or CH;
Xₐ is N or CR₃ₐ;
R₁ₐ is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R₂ₐ is -(L)ₘ-S(O)OH, -L-(CH₂)ₙS(O)OH, -(L)ₘ-S(O)₂OH, -L-(CH₂)ₙS(O)₂OH;
m is 0 or 1;
n is 0 or 1;
L is CH₂, CHF, or CF₂;
R₃ₐ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R₄ₐ is cyclopropyl, cyclobutyl, or —C₁₋₃ alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof.

18. The compound of claim 2, wherein the compound has a structure represented by Formula B2: 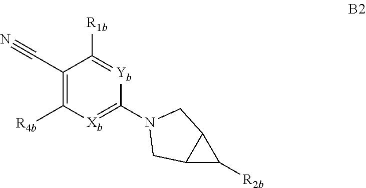
wherein
Y_{b} is N or CH;
Xb is N or CR_{3b};
R_{1b} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R_{2b} is -(L)ₘ-S(O)OH, -L-(CH₂)ₙS(O)OH, -(L)ₘ-S(O)₂OH or -L-(CH₂)ₙS(O)₂OH;
m is 0 or 1;
n is 0 or 1;
L is CH₂, CHF, or CF₂;
R_{3b} is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R_{4b} is cyclopropyl, cyclobutyl, or —C₁₋₃alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof.

19. The compound of claim 2, wherein the compound has a structure represented by Formula B3: 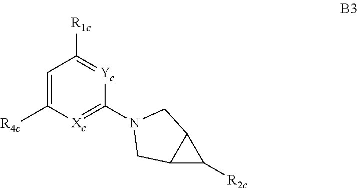
wherein
Y_{c} is N or CH;
X_{c} is N or CR_{3c};
R_{1c} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl and —OH, wherein —C₁₋₃ alkyl is substituted with 0 to 3 halogen atoms, and provided that there is no more than one —OH substituent; or
—N(C₁₋₃ alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 OH;
R_{2c} is -(L)ₘ-S(O)OH, -L-(CH₂)ₙS(O)OH, -(L)ₘ-S(O)₂OH or -L-(CH₂)ₙS(O)₂OH;
m is 0 or 1;
n is 0 or 1;
L is CH₂, CHF, or CF₂;
R₃ is H, halogen, —CN, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms; and
R_{4c} is cyclopropyl, cyclobutyl, or —C₁₋₃alkyl, wherein the C₁₋₃ alkyl is optionally substituted with 0 to 5 halogen atoms;
or an enantiomer, stereoisomer, tautomer, solvate, hydrate, amino acid conjugate, or pharmaceutically acceptable salt thereof.

20. The compound of claim 6, wherein the compound has a structure represented by Formula C1: 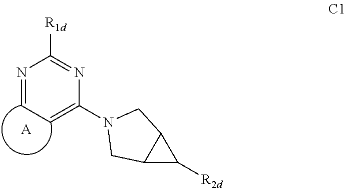
wherein
Ring A is a C₄₋₇ cycloalkyl ring wherein each carbon atom in the ring is substituted by 0 to 2 R5 groups
R_{1d} is C₃₋₇ cycloalkyl or a 4- to 7-membered heterocyclic moiety, wherein the heterocyclic moiety contains 1 to 2 atoms independently selected from nitrogen, oxygen and sulfur, and wherein the cycloalkyl or heterocyclic moiety has 0 to 3 substituents independently selected from —C₁₋₃ alkyl, F and —OH, wherein —C₁₋₃ alkyl is substituted with 0 to 3 halogen atoms, and provided that there is no more than one —OH substituent; or
R_{1d} is —N(C₁₋₃alkyl)₂, —NH(C₁₋₃ alkyl), or —NH(C₃₋₄ cycloalkyl), wherein each C₁₋₃ alkyl or C₃₋₄ cycloalkyl is substituted with 0 to 1 —OH substituent;
R_{2d} is -(L)ₘ-S(O)OH, -L-(CH₂)ₙS(O)OH, -(L)ₘ-S(O)₂OH, -L-(CH₂)ₙS(O)₂OH;
m is 0 or 1;
n is 0 or 1;
L is CH₂, CHF, or CF₂;
R_{5d} is F, —C₁₋₃ alkyl, —OC₁₋₃ alkyl, —C₃₋₄ cycloalkyl, wherein each C₁₋₃ alkyl or —C₃₋₄ cycloalkyl is optionally substituted with 1 to 3 halogen atoms;
or an enantiomer, stereoisomer, tautomer, solvate, hydrate, amino acid conjugate, or pharmaceutically acceptable salt thereof.
